(19) United States
(12) Patent Application Publication
(10) Pub. No.: US 2021/0172940 A1
(43) Pub. Date: Jun. 10, 2021

(54) Photothermal Effects-Driven Volumetric Bar-Chart Microchip

(71) Applicant: Board of Regents, The University of Texas System, Austin, TX (US)

(72) Inventors: XiuJun Li, El Paso, TX (US); Guanglei Fu, El Paso, TX (US); Wan Zhou, El Paso, TX (US)

(21) Appl. No.: 17/112,056

(22) Filed: Dec. 4, 2020

Related U.S. Application Data
(60) Provisional application No. 62/944,270, filed on Dec. 5, 2019.

(51) Int. Cl. G01N 33/543; G01N 33/558; B01L 3/00

(52) U.S. Cl. CPC G01N 33/54366; G01N 33/558; B01L 2400/0442; B01L 3/50273; B01L 2200/0605; G01N 33/54346

(57) ABSTRACT

A type of microfluidic platform, photothermal bar-chart chip (PT-Chip), uses on-chip nanomaterial-mediated photothermal effect as a tunable microfluidic driving force to drive ink bar-charts in a visual quantitative readout fashion. The photothermal bar-chart pumping performance can be adjusted remotely by tuning the irradiation parameters, without the need to change any on-chip parameters. The PT-Chip enables a POC visual quantitative diagnostics, by forming nanomaterial-mediated photothermal effects-driven bar-chart microchip for visual quantitative immuno-sensing. In this immunoassay, biomolecules are visually quantified by directly reading the distance that fluids move on the PT-Chip.

### CROSS REFERENCE TO RELATED APPLICATIONS

This application is a utility conversion and claims priority to U.S. Ser. No. 62/944,270, filed Dec. 5, 2019, the contents of which are incorporated herein by reference in their entirety for all purposes.

### BACKGROUND INFORMATION

### 1. Field

The present invention relates generally to the field of medicine and disease diagnosis. More particularly, it concerns kits and devices for detecting disease using a photothermal immunoassay.

### 2. Background

Biomarkers have been perceived as an indicator of normal biological or pathogenic processes, or pharmacological responses to a therapeutic invention. Since the 1980s, the detection of biomarkers has been widely developed and played an important role in disease diagnoses, such as cancer and heart disease. In cancer research, cancer biomarkers have played an important role in screening, disease diagnosis, prognosis, and treatment, which are used to provide crucial information to guide prognostic and therapeutic decisions. Currently, various immunoassay methods for the detection of biomarkers include surface plasmon resonance (SPR), colorimetry, fluorescence, electrochemistry, and chemiluminescence. Unfortunately, most conventional methods have posed limitations to the widely point-of-care (POC) application, especially in resource-limited settings. Particularly, the methods with high sensitivity (e.g., SPR, fluorescence, and electrochemistry) are relying on expensive biochemicals (e.g. enzymes), complicated immune procedures, as well as costly and bulky analytical instruments (e.g. fluorescence microscopy and microplate readers). Therefore, there has been a sustained need for developing novel immunoassay strategies for the low-cost, simple, and portable POC detection of biomarkers.

Microfluidics has emerged as an increasingly attractive technology for POC testing (POCT), especially in the fields of medical diagnostics, food and drug safety inspection, and environmental surveillance, because of their outstanding merits of affordability, simplicity, portability, and high-throughput measurement. Despite great research progress in microfluidics after more than two decades of development, microfluidic platforms are still confronted with several major challenges. In particular, the requirement of external microfluidic pumping accessories, such as pneumatic syringe pumps, usually results in higher cost, additional space, and operational complexity. Additionally, the assay readouts of these microfluidic chips usually rely on bulky and costly instruments and detectors, such as fluorescence microscopes, electrochemical working stations, and microplate readers. All these pumping and detection accessories have significantly compromised the inherent advantages of high portability and integrability and low cost from microfluidic systems.

Microfluidic lab-on-a-chip (LOC) technology provides a great opportunity for the detection of biomarkers, with numerous benefits including low reagent consumption, miniaturization, integration, and portability. The volumetric bar-chart microfluidic chips (V-Chips), as one of the POC devices, provide a simple yet powerful platform for visual biochemical quantitation. Based on the volumetric measurement of enzyme- or nanoparticle-catalyzed gas production that acts as the microfluidic pump of the dye movement, different types of V-Chips have been developed for quantitative detection of various disease biomarkers and pathogens.

However, several limitations are usually encountered in these V-Chips, such as relatively low operational stability because of inevitable denaturation of enzymes, and dependence of the catalytic activity on surrounding environments. In addition, imprecise spatiotemporal controllability of the catalytic reaction and limited pumping strength of the gas-production approach remain major challenges due to intrinsic properties of the enzyme- or nanoparticle-catalyzed reactions. Therefore, more robust, stable, on-demand, adjustable, and multiplexed microfluidic pumping systems are highly needed to be integrated into bar-chart chips.

### SUMMARY

An illustrative embodiment provides a new type of microfluidic platforms, photothermal bar-chart chip (PT-Chip), has been developed using the on-chip nanomaterial-mediated photothermal effect as the novel tunable microfluidic driving force to drive ink bar-charts in a visual quantitative readout fashion. The photothermal bar-chart pumping performance can be adjusted remotely by tuning the irradiation parameters, without the need to change any on-chip parameters, such as enzyme concentrations in conventional immunoassays.

An illustrative embodiment provides an improved V-chip driven for POC visual quantitative diagnostics, forming nanomaterial-mediated photothermal effects-driven volumetric bar-chart microchip (PT-Chip) for visual quantitative immuno-sensing. In this immunoassay, biomolecules were visually quantified by directly reading the distance that fluids moved on the PT-chip, without the aid of bulky and expensive analytical instruments.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B depict a schematic illustration of (A) the photothermal bar-chart chip using the on-chip nanomaterial-mediated photothermal effect as the microfluidic driving force and (B) multiplexed on-chip transport of substances using the photothermal microfluidic pump;

FIG. 2 depicts optical absorption properties of nanomaterials and dye indicators in the NIR region. UV-Vis absorption spectra of methylene blue (MB, 0.0125 mg/mL), red food dye (RD), PB NPs (0.1 mg/mL) and GO (0.1 mg/mL) aqueous suspensions;

FIGS. 3A-3D depict off-chip photothermal effect-induced temperature elevation of nanomaterials and dyes as a function of (A-B) irradiation time and (C-D) nanomaterial concentration. Temperature comparison among (A) different concentrations of PB NPs suspended in MB solutions (0.054 mg/mL), and (B) different concentrations of GO suspended in RD solutions during the laser irradiation process for 10 min at a power density of 3.12 W·cm-2. Temperature increases (ΔT) vs. concentrations of (C) PB NPs suspensions and (D) GO suspensions after the laser irradiation for 1.0 min at a power density of 5.26 W·cm-2. Error bars represent standard deviations (n=4);

FIGS. 4A-4C depict a schematic illustration of structures of photothermal bar-chart Chip 1, Chip 2, and Chip 3;

FIGS. 5A-5B depict on-chip photothermal bar-chart microfluidic pumping performance on the PT-Chip (Chip 1). (a) Photographs of the PT-Chip loaded with blank MB, PB NPs-MB (0.1 mg/mL), blank RD, and GO-RD (1.0 mg/mL) suspensions before and after the laser irradiation for 50 s at a power density of 2.6 W·cm-2. (b) Comparison among on-chip bar-chart pumping distances after the laser irradiation. Error bars represent standard deviations (n=4);

FIGS. 6A-6D depict an effect of laser irradiation time on photothermal bar-chart microfluidic pumping performance on the PT-Chip (Chip 1). (A) Photographs of the PT-Chip loaded with PB NPs-MB suspensions (0.1 mg/mL) after the laser irradiation for different times at 2.6 W·cm-2. (B) On-chip bar-chart pumping distance of the PB NPs-MB suspensions as a function of the irradiation time. (C) Photographs of the PT-Chip loaded with GO-RD suspensions (1.0 mg/mL) after the laser irradiation for different times. (D) Bar-chart pumping distance of the GO-RD suspensions as a function of the irradiation time. Error bars represent standard deviations (n=4);

FIGS. 7A-7D depict an effect of nanomaterial concentration on photothermal bar-chart microfluidic pumping performance on the PT-Chip (Chip 1). (A) Photographs of the PT-Chip loaded with different concentrations of PB NPs-MB suspensions after the laser irradiation for 50 s at 2.6 W·cm-2. (B) Calibration plot of bar-chart pumping distance of the PB NPs-MB suspensions vs. PB NPs concentration. (C) Photographs of the PT-Chip loaded with different concentrations of GO-RD suspensions after the laser irradiation for 90 s. (D) Calibration plot of bar-chart pumping distance of the GO-RD suspensions vs. GO concentration. Error bars represent standard deviations (n=4);

FIGS. 8A-8D depict reproducibility of photothermal bar-chart microfluidic pumping performance on the PT-Chip. (A) Photographs and (B) the measured bar-char pumping distance of Chip 1 loaded with the same concentration of PB NPs-MB suspensions (0.05 mg/mL) after the laser irradiation for 50 s at 2.6 W·cm-2. (C) Photographs and (D) the measured bar-chart pumping distance of Chip 1 loaded with the same concentration of GO-RD suspensions (0.5 mg/mL) after the laser irradiation for 90 s′;

FIGS. 9A-9B depict application of the photothermal microfluidic pump for on-chip transport of dyes and Au NPs. (A) On-chip transport rate as a function of PB NPs concentrations in the single-sample transport PT-Chip (Chip 2). Insets: Photographs of Chip 2 before and after the laser irradiation (2.6 W·cm-2) at a PB NPs concentration of 0.1 mg/mL. (B) Photographs of the multiplexed transport PT-Chip (Chip 3) before and after the laser irradiation at a PB NPs concentration of 0.4 mg/mL. Error bars represent standard deviations (n=4).

FIGS. 10A-10B depict the PDMS/PMMA hybrid PT-Chip (Chip 4) (A) The designed bottom PMMA layer, and (B) three layers assembly design;

FIG. 11 depicts a transformation reaction from Fe₃O₄ NPs to PB NPs and the general structure of PB NPs;

FIG. 12 depicts working principle of PT-Chip for the visual quantitative detection of PSA based on the nanomaterials-mediated photothermal effect;

FIGS. 13A-13B depict characterization of the nanoparticles-mediated immuno-sensing process. (A) UV-vis spectra and the photographs of immuno-sensing solution after the transformation from Fe₃O₄ NPs to PB NPs at different PSA concentrations. (B) Calibration plot of absorbance at 748 nm vs. logarithm of the PSA concentration. Error bars represent standard deviations (n=3).

FIGS. 14A-14B depict visual quantitative detection of PSA using the PDMS/PMMA hybrid PT-Chip. (A) Photographs of on-chip detection at different concentrations of PSA in PBS buffer under the laser irradiation at 5 min. Four parallel experiments were conducted on a single chip. Red arrows indicate the end location of bar-charts. (B) Calibration plot of moving distances (ΔLs) vs. the logarithmical concentrations of standard PSA. Error bars represent standard deviations (n=4). (1 a.u.=2.0 mm, laser power 2.21 W/cm²);

FIGS. 15A-15B depict visual quantitative detection of PSA using the PDMS/PMMA hybrid PT-Chip. (A) Photographs of on-chip detection at different concentrations of PSA in 5-fold diluted normal human serum samples under the laser irradiation at 5 min. Four parallel experiments were conducted on a single chip. Red arrows indicate the end location of bar-charts. (B) Calibration plot of moving distances (ΔLs) vs. the logarithmical concentrations of spiking PSA. Error bars represent standard deviations (n=4). (1 a.u.=2.0 mm, laser power 2.21 W/cm²); and

FIG. 16 depicts a specificity study. Moving distances recorded for the detection of PSA (4 ng/mL and 8 ng/mL) in PBS buffer, 5-fold diluted human serum, and 10-fold diluted fresh human whole blood samples. Other interfering substances were tested including HBsAg, CEA, and IgG with the spiking concentration of 80 ng/mL, in which PBS buffer as blank. Error bars represent standard deviations (n=4).

### DETAILED DESCRIPTION

According to illustrative examples described herein, a photothermal bar-chart chip (PT-Chip), uses the on-chip nanomaterial-mediated photothermal effect as the microfluidic driving force. The nanomaterial-mediated photothermal effect is employed as the microfluidic pump in a PT-Chip to propel on-chip ink-bar-chart movement in a visual quantitative readout fashion. Upon the contact-free irradiation of the nanomaterial suspensions on the PT-Chip by a near infrared laser, the on-chip photothermal effect results in rapid and substantial heat production. The subsequent heating of solutions leads to the rapid accumulation of vapor pressure in limited volumes of the inlets, thereby pumping the on-chip visual bar-chart movement of the nanomaterial suspensions. In certain aspects, the PT-Chip can be employed for multiplexed on-chip transport of substances, such as gold nanoparticles (Au NPs).

In another illustrative example, iron oxide nanoparticles (Fe₃O₄ NPs)-mediated photothermal effect-driven volumetric bar-chart microchip (PT-Chip) enables visual quantitative immuno-sensing. In this strategy, Fe₃O₄ NPs involved in a typical sandwich immunoassay were converted to Prussian blue nanoparticles (PB NPs), a NIR photothermal agent. PB NPs were exploited to generate heat under the laser irradiation, acting as a powerful driving force on the chip. As such, the immuno-sensing signals were converted to visual bar-charts on the PT-Chip. The quantitation of biomolecules was achieved by visually reading the colored flowing distance on the PT-Chip, without the aid of any bulky and expensive instruments. The exploration of the PT-Chip provides unprecedented advances for the POC detection of biomarkers and opens a new horizon of microfluidic LOC devices for broad applications.

The photothermal agent is not particularly limited as long as it is able to convert energy of light into thermal energy. In certain aspects, a photothermal agent is a nanomaterial, dye, or pigment that absorb certain wavelengths of light and convert the absorbed light into heat. The dye may be, but is not limited to azo dyes, metal complex salt azo dyes, pyrazolone azo dyes, naphthoquinone dyes, anthraquinone dyes, phthalocyanine dyes, carbonium dyes, quinonimine dyes, methine dyes, cyanine dyes, squarylium pigments, pyrylium salts, and metal thiolate complex. Examples of pigments include, but are not limited to black pigments, yellow pigments, orange pigments, brown pigments, red pigments, violet pigments, blue pigments, green pigments, fluorescent pigments, metallic powder pigments, and other pigments such as polymer-binding pigments. Specifically, it is possible to use insoluble azo pigments, azo lake pigments, condensed azo pigments, chelate azo pigments, phthalocyanine type pigments, anthraquinone type pigments, perylene and perinone type pigments, thioindigo type pigments, quinacridone type pigments, dioxazine type pigments, isoindolinone type pigments, quinophthalone type pigments, dyed lake pigments, azine pigments, nitroso pigments, nitro pigments, natural pigments, fluorescent pigments, inorganic pigments, carbon black, or the like. In certain aspects the photothermal agent is iron oxide nanoparticles, Prussian blue nanoparticles, the charge transfer complex of the one-electron oxidation product of TMB (oxidized TMB), gold nanorods, graphene oxide, carbon nanotubes, Indocyanine Green, CuS-based nanomaterials, or other photothermal nanomaterials.

The term “nanomaterial” as used herein, refers to particles comprising at least an iron oxide core or other materials with at least one dimension in the range of about 1 to about 1,000 nanometers (“nm”). The nanomaterials of the invention may be of any shape. In certain embodiments the nanoparticles are spherical. The nanoparticles of the invention typically do not, but can, include a light-active molecule.

The nanomaterials of the invention may be chemically transformed to nanoparticles that enhance the conversion of light to heat. The surface of the nanoparticle may be coupled directly or indirectly with a light absorbing moiety. In some embodiments, the surface of the nanoparticle is treated or derivatized to permit attaching a ligand to the surface of the nanoparticle.

The phrase “increases the thermal activity of the nanomaterial” means exposure to a light source of the appropriate wavelength results in a nanoparticle providing increased signal or sensitivity when measured by color or heat in, for example, an immunoassay, as compared to a non-thermal active nanoparticle.

As used herein, “ligand” means a molecule of any type that will bind to an analyte of interest. For example and without limitation, in certain embodiments the ligand is an antibody, an antigen, a receptor, a nucleic acid, or an enzyme.

The term “analyte” as used herein refers to any substance of interest that one may want to detect using the invention, including but not limited to drugs, including therapeutic drugs and drugs of abuse; hormones; vitamins; proteins, including antibodies of all classes; peptides; steroids; bacteria; fungi; viruses; parasites; components or products of bacteria, fungi, viruses, or parasites; allergens of all types; products or components of normal or malignant cells; etc. in certain embodiments of the invention, the presence or absence of an analyte in a sample is determined qualitatively. In other embodiments, the amount or concentration of analyte in the sample is quantitatively determined.

The term “sample” as used herein refers to any biological sample that could contain an analyte for detection. In some embodiments, the biological sample is in liquid form, while in others it can be changed into a liquid form.

As used herein in the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The terms “approximately”, “about”, and “substantially” as used herein to represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

Analytes can be detected using the photothermal methodologies described herein in a variety of assays including, but not limited to immuno-detection, microchip, or lateral flow based methods. In certain aspects the analyte detection methods employ an analyte specific ELISA assay. In certain aspects, antibodies directly or indirectly coupled to a thermogenic nanoparticle (a nanoparticle that is coupled to, can be transformed into, or catalyzes the production of a photothermal agent) are used to detect the presence of an analyte in an original or processed sample. In certain aspects the sample is a biological sample obtained from a subject. Samples obtained from a subject may include, for example, cells, tissue, blood, serum, or urine. For example, a sample can be blood or urine collected from a subject. A sample can be analyzed directly or extracted/processed before analysis.

In certain aspects, a sample is contacted with an effective amount of one or more binding agents that specifically binds the target analyte to form a complex. The complex or binding reaction is then detected directly when the binding reagent is coupled to a thermogenic agent or indirectly by contacting the complex with a second thermogenic agent that specifically binds the complex or the binding reagent, or the analyte present in the complex. In certain embodiments the binding reagent is an antibody or antibody fragment. The antibody can be coupled to a thermogenic agent, such as a NP as described herein.

In other embodiments, the analyte in the sample is immobilized on a surface and detected. In certain aspects, analyte is immobilized prior to introduction of the thermogenic agent, and the amount of the signal, corresponding to the amount of thermogenic agent bound, correlates to the amount of analyte in the sample. In still other embodiments, the analyte is captured by an immobilized unlabeled first binding reagent, after which a thermogenic second agent is introduced to bind to the captured analyte and produce a signal in proportion to the amount of captured analyte.

A thermogenic agent can be coupled to a first antibody and used as a binding agent in a direct assay or coupled to a secondary antibody to detect a first preformed antibody/analyte complex in an indirect assay. Additionally, an antibody can be used in a competition assay to detect analytes in a sample. For example, analytes in a sample are captured by an unlabeled antibody immobilized on the surface of an ELISA well and then detected by a labeled (thermogenic) antibody of the same or different kind and/or specificity. Alternatively, the sample can be suspended in a buffer and mixed directly with an antibody, thus allowing the antibody to form an immune complex with the analyte. The reduction of free antibody due to complex formation can then be determined in a second step, based on solid-phase ELISA with purified analytes by comparing the relative reactivity of free residual antibody left over after sample incubation (sample reactivity) to that of the same antibody when not mixed with the sample (reference reactivity). The ratio of sample to reference antibody reactivity will be inversely proportional to the amount of analyte in the sample.

In certain aspects, methods of the invention can be adapted for lateral flow assays and other immunoassays and devices supporting such assays. Lateral flow assays, also known as immunochromatographic assays, are typically carried out using a simple device intended to detect the presence (or absence) of a target analyte in the sample. Most commonly these tests are used for medical diagnostics either for home testing, point of care testing, or laboratory use. Often produced in a dipstick format, these assays are a form of immunoassay in which the test sample flows along a solid substrate via capillary action. After the sample is applied to the test it encounters a colored or labeling reagent (thermogenic agent) which mixes with the sample and transits the substrate encountering lines or zones which have been pretreated with an antibody or antigen or affinity reagent. Depending upon the analyte present in the sample the colored or labeling reagent can become bound at the test line or zone. Lateral flow assays can operate as either competitive or sandwich assays.

As used herein, the term “carrier,” such as used in a lateral flow assay, refers to any substrate capable of providing liquid flow. This would include, for example, substrates such as nitrocellulose, nitrocellulose blends with polyester or cellulose, untreated paper, porous paper, rayon, glass fiber, acrylonitrile copolymer, plastic, glass, or nylon. The substrate may be porous. Typically, the pores of the substrate are of sufficient size such that the nanoparticles of the invention flow through the entirety of the carrier. One skilled in the art will be aware of other materials that allow liquid flow. The carrier may comprise one or more substrates in fluid communication. For example, the reagent zone and detection zone may be present on the same substrate (i.e., pad) or may be present on separate substrates (i.e., pads) within the carrier.

As used herein, “porous membrane,” such as used in a flow through assay, refers to a membrane or filter of any material that wets readily with an aqueous solution and has pores sufficient to allow nanoparticles of the invention to pass through. Suitable materials include, for example, nitrocellulose, nitrocellulose blends with polyester or cellulose, untreated paper, porous paper, rayon, glass fiber, acrylonitrile copolymer, plastic, glass, or nylon.

As used herein, “absorbent material” refers to a porous material having an absorbing capacity sufficient to absorb substantially all the liquids of the assay reagents and any wash solutions and, optionally, to initiate capillary action and draw the assay liquids through the test device. Suitable materials include, for example, nitrocellulose, nitrocellulose blends with polyester or cellulose, untreated paper, porous paper, rayon, glass fiber, acrylonitrile copolymer, plastic, glass, or nylon.

As used herein, the term “lateral flow” refers to liquid flow along the plane of a carrier. In general, lateral flow devices may comprise a strip (or several strips in fluid communication) of material capable of transporting a solution by capillary action, i.e., a wicking or chromatographic action, wherein different areas or zones in the strip(s) contain assay reagents either diffusively or non-diffusively bound that produce a detectable signal as the solution is transported to or through such zones. Typically, such assays comprise an application zone adapted to receive a liquid sample, a reagent zone spaced laterally from and in fluid communication with the application zone, and a detection zone spaced laterally from and in fluid communication with the reagent zone. The reagent zone may comprise a compound that is mobile in the liquid and capable of interacting with an analyte in the sample and/or with a molecule bound in the detection zone. The detection zone may comprise a binding molecule that is immobilized on the strip and is capable of interacting with the analyte and/or the reagent compound to produce a detectable signal. Such assays may be used to detect an analyte in a sample through direct (sandwich assay) or competitive binding.

In a sandwich lateral flow assay, a liquid sample that may or may not contain an analyte of interest is applied to the application zone and allowed to pass into the reagent zone by capillary action. The analyte, if present, interacts with a labeled reagent in the reagent zone and the analyte-reagent complex moves by capillary action to the detection zone. The analyte-reagent complex becomes trapped in the detection zone by interacting with a binding molecule specific for the analyte and/or reagent. Unbound sample may move through the detection zone by capillary action to an absorbent pad laterally juxtaposed and in fluid communication with the detection zone. The labeled reagent may then be detected in the detection zone by appropriate means.

In a competitive lateral flow assay, a liquid sample that may or may not contain an analyte of interest is applied to the application zone and allowed to pass into the reagent zone by capillary action. The reagent zone comprises a labeled reagent, which may be the analyte itself, a homologue or derivative thereof, or a moiety that is capable of mimicking the analyte of interest when binding to an immobilized binder in the detection zone. The labeled reagent is mobile in the liquid phase and moves with the liquid sample to the detection zone by capillary action. The analyte contained in the liquid sample competes with the labeled reagent in binding to the immobilized binder in the detection zone. Unbound sample may move through the detection zone by capillary action to an absorbent pad laterally juxtaposed and in fluid communication with the detection zone. The labeled reagent may then be detected in the detection zone by appropriate means. The presence or absence of the analyte of interest may be determined through inspection of the detection zone, wherein the greater the amount of analyte present in the liquid sample, the lesser the amount of labeled receptor bound in the detection zone.

As used herein, the terms “vertical flow” and “flow through” refer to liquid flow transverse to the plane of a carrier. In general, flow through devices may comprise a membrane or layers of membranes stacked on top of each other that allow the passage of liquid through the device. The layers may contain assay reagents either diffusively or non-diffusively bound that produce a detectable signal as the solution is transported through the device. Typically, the device comprises first layer having an upper and lower surface, wherein said upper surface is adapted to receive a liquid sample, and an absorbent layer vertically juxtaposed and in fluid communication with the lower surface of the first layer that is adapted to draw the liquid sample through the first layer. The first layer may comprise a binding agent attached to the upper surface of the first layer that is capable of interacting with an analyte in the sample and trapping the analyte on the upper surface of the first layer.

In practice, a liquid sample that may or may not contain an analyte of interest is applied to the upper surface of a first layer comprising a binding agent specific for an analyte of interest. The liquid sample then flows through the first layer and into the absorbent layer. If analyte is present in the sample, it interacts with the binding agent and is trapped on the upper surface of the first layer. The first layer may then be treated with wash solutions in accordance with conventional immunoassay procedures. The first layer may then be treated with a labeled reagent that binds to the analyte trapped by the binding agent. The labeled reagent then flows through the first layer and into the absorbent layer. The first layer may be treated with wash solutions in accordance with conventional immunoassay procedures. The labeled reagent may then be detected by appropriate means. Alternatively, the liquid sample may be mixed with the labeled reagent before being applied to the upper surface of the first layer. Other suitable variations are known to those skilled in the art.

Lateral and flow through assays may be used to detect multiple analytes in a sample. For example, in a lateral flow assay, the reagent zone may comprise multiple labeled reagents, each capable of binding to (or mimicking) a different analyte in a liquid sample, or a single labeled reagent capable of binding to (or mimicking) multiple analytes. Alternatively, or in addition, the detection zone in a lateral flow assay may comprise multiple binding molecules, each capable of binding to a different analyte in a liquid sample, or a single binding molecule capable of binding to multiple analytes. In a flow through assay, the porous membrane may comprise multiple binding agents, each capable of binding to a different analyte in a liquid sample, or a single binding agent capable of binding to multiple analytes. Alternatively, or in addition, a mixture of labeled reagents may be used in a flow through assay, each configured to bind to a different analyte in a liquid sample, or a single labeled reagent configured bind multiple analytes. If multiple labeled reagents are used in a lateral or flow through assay, the reagents may be differentially labeled to distinguish different types of analytes in a liquid sample.

As used herein, the term “mobile” means diffusively or non-diffusively attached, or impregnated. The reagents which are mobile are capable of dispersing with the liquid sample and are carried by the liquid sample in the lateral or vertical flow.

As used herein, the term “labeled reagent” means any particle, protein, or molecule which recognizes or binds to the analyte of interest and has attached to it a substance capable of producing a signal that is detectable visually in a volumetric bar-chart microfluidic chip, that is, a thermogenic nanomaterial as defined herein. The particle or molecule recognizing the analyte can be either natural or non-natural. In some embodiments the molecule is a monoclonal or polyclonal antibody.

As used herein, the term “binding reagent” means any particle or molecule which recognizes or binds a target analyte. The binding reagent is capable of forming a binding complex with the analyte-labeled reagent complex. The binding reagent can be immobilized to a carrier in the detection zone or to the surface of a membrane or support. The particle or molecule can be natural, or non-natural, e.g., synthetic.

As used herein, the term “detection zone” means the portion of the carrier or support containing an immobilized binding reagent.

The term “control zone” refers to a portion of the test device comprising a binding molecule configured to capture the labeled reagent. In a lateral flow assay, the control zone may be in liquid flow contact with the detection zone of the carrier, such that the labeled reagent is captured in the control zone as the liquid sample is transported out of the detection zone by capillary action. In a flow through assay, the control zone may be a separate portion of the porous membrane, such that the labeled reagent is applied both to the sample application portion of the porous membrane and the control zone. Detection of the labeled reagent in the control zone confirms that the assay is functioning for its intended purpose.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

The inventors provide a solution to the need for easy-to-use detection devices for point-of-care (POC) detection and assay. The inventors describe herein a photothermal immunoassay that meets the needs of a POC device. The concept of photothermal conversion has emerged as a particularly attractive research topic in various fields because of the unique light-to-heat photo-physical conversion property. In particular, near-infrared (NIR) light-driven photothermal conversion has been intensively applied in biomedical field for photothermal therapy of cancers employing heat converted by photothermal agents from NIR light absorption.

### 3. PHOTOTHERMAL BAR-CHART MICROFLUIDIC PLATFORM

Embodiments of the invention are directed to nanomaterial-based photothermal immunoassays employing a volumetric bar-chart microfluidic chip for sensitive quantitative readout of analyte levels based on a photothermal strategy.

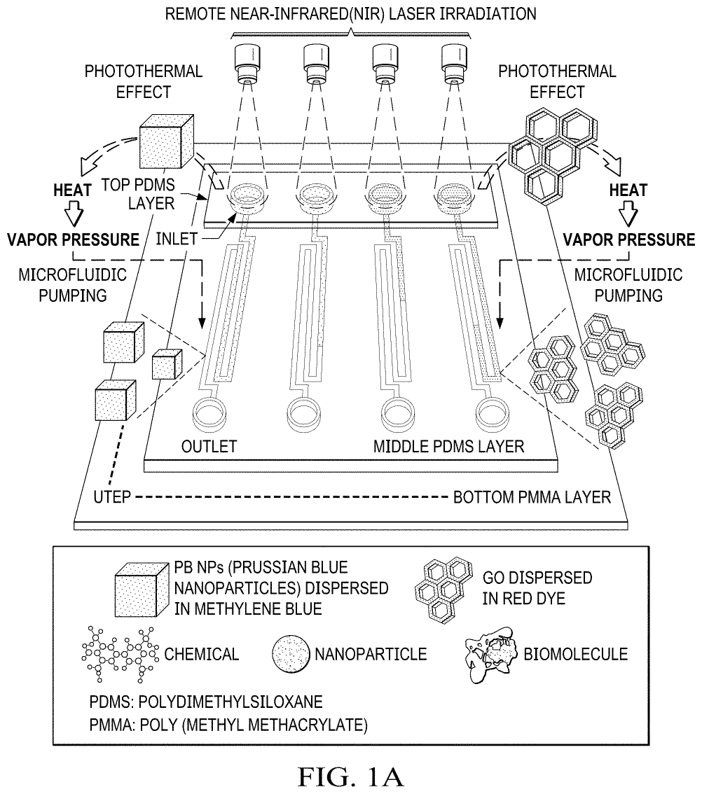

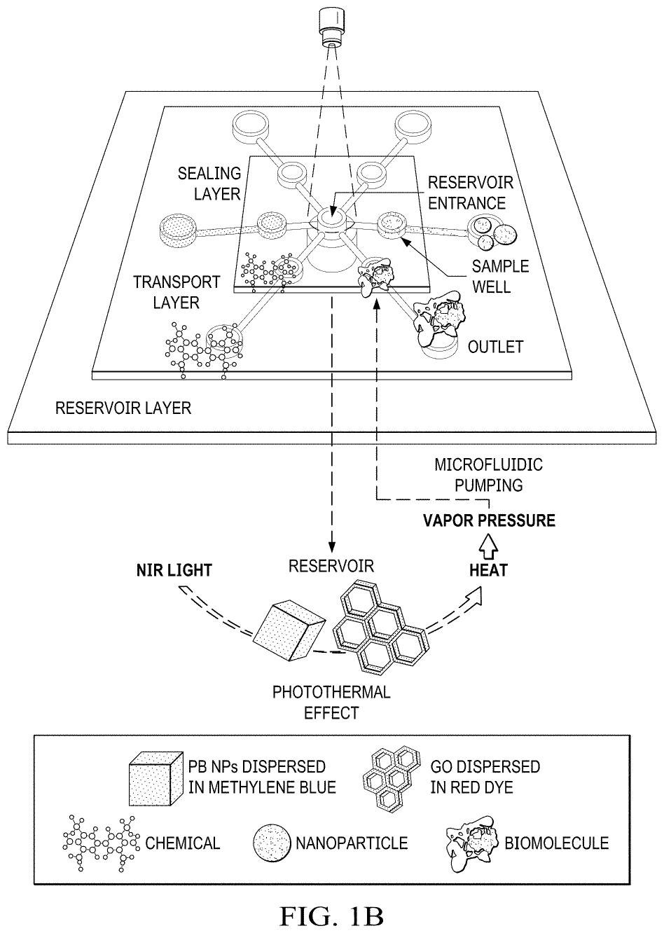

As illustrated schematically in FIG. 1, photothermal bar-chart chip (PT-Chip) uses the on-chip nanomaterial-mediated photothermal effect as the novel tunable microfluidic driving force to drive ink bar-charts in a visual quantitative readout fashion. The photothermal bar-chart pumping performance can be adjusted remotely by tuning the irradiation parameters, without the need to change any on-chip parameters, such as enzyme concentrations. In contrast to graphene oxide, Prussian blue nanoparticles with stronger photothermal conversion efficiency were used as the model photothermal agent to demonstrate the proof of concept. Upon the contact-free irradiation by an 808 nm laser pointer, the strong on-chip nanomaterial-mediated photothermal effect can serve as a robust, remotely tunable, and stable microfluidic pump in a PMMA/PDMS hybrid bar-chart chip. The on-chip pumping distance of the ink bars is linearly correlated with both the irradiation time and the nanomaterial concentration. The application of the photothermal pump is exemplified for multiplexed on-chip transport of substances, such as gold NPs. This is the first report of the PT microfluidic platform, which has great potential for various microfluidic applications.

In another illustrative example, iron oxide nanoparticles (Fe₃O₄ NPs)-mediated photothermal effect-driven volumetric bar-chart microchip (PT-Chip) enables visual quantitative immuno-sensing. In this strategy, Fe₃O₄ NPs involved in a typical sandwich immunoassay were converted to Prussian blue nanoparticles (PB NPs), a NIR photothermal agent. PB NPs were exploited to generate heat under the laser irradiation, acting as a powerful driving force on the chip. As such, the immuno-sensing signals were converted to visual bar-charts on the PT-Chip. The quantitation of biomolecules was achieved by visually reading the colored flowing distance on the PT-Chip, without the aid of any bulky and expensive instruments. In this illustrative example, this PT-Chip was employed to detect the prostate-specific antigen (PSA) with high specificity and sensitivity. The obtained LOD of 2.0 ng/mL could meet the clinical requirement of PSA testing to identify the early stage of prostate cancer. This method was further validated by testing human serum and fresh whole blood samples, with satisfactory analytical recoveries in the range from 89.1% to 92.5%. The exploration of the PT-Chip provides unprecedented advances for the POC detection of biomarkers and opens a new horizon of microfluidic LOC devices for broad applications.

### 4. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### 4.A. Example 1: Nanomaterial-Mediated Photothermal Effect as Microfluidic Driving Force

In one illustrative example, a new type of microfluidic platforms is provided, photothermal bar-chart chip (PT-Chip), based on the integration of on-chip nanomaterial-mediated photothermal effect as the novel on-demand microfluidic driving force.

During the irradiation process of the nanomaterial-dye suspensions, the on-chip nanomaterial-mediated photothermal effect resulted in rapid and substantial heat production in the inlets. The subsequent heating of solutions led to the rapid accumulation of vapor pressure inside the inlets with limited volumes, thereby driving the visual bar-chart movement of the nanomaterial-dye suspensions in the channels. Therefore, a new type of bar-chart microfluidic platform, PT-Chip, was developed using the on-chip nanomaterial-mediated photothermal effect as the microfluidic driving force, as illustrated in FIG. 1. In addition to the robustness of the photothermal microfluidic pump, another key feature of the new PT-Chip is that the on-chip bar-chart pumping rate is highly tunable by remotely adjusting the parameters of the laser, whereas it is quite challenging to spatiotemporally adjust enzyme- or nanoparticle-catalyzed reactions on a chip. It was observed that the progressive bar-chart movement can be instantly terminated upon the removal of the laser irradiation. It should be noted that the nanomaterial-mediated photothermal effect is more stable than the enzyme-catalyzed gas-production approach as the microfluidic bar-chart pump, because enzymes can easily lose activity at room temperature while the photo-physical conversion efficiency is intrinsically inert to surrounding environments. Furthermore, by tuning the irradiation diameter of the laser spot and changing the layout of the chip, multiple inlets can be irradiated at one irradiation process, enabling on-demand microfluidic pumping in a multiplexed manner. These results indicated the feasibility to utilize the on-chip nanomaterial-mediate photothermal effect as a new type of robust, tunable and versatile microfluidic pumps in bar-chart chips.

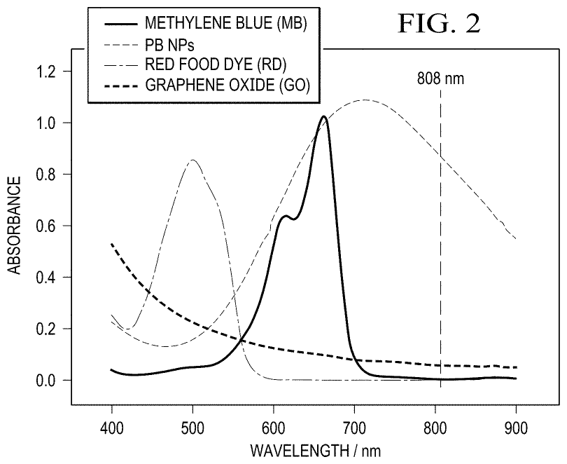

As shown in FIG. 2, three (3) different chips illustrate different fundamental aspects and demonstrated the proof of concept of the tunable PT-Chip. The remotely tunable photothermal effect of nanomaterials propelled robust on-chip bar-chart movement of dyes, enabling visual quantitative readouts and the exploration of on-chip nanomaterial-mediated photothermal effect as a new type of on-demand microfluidic pumps. The application of the photothermal microfluidic pump is exemplified for on-chip transport of substances, such as Au NPs, by using Chip 2 and Chip 3 in a multiplexed manner. In comparison with the traditional V-Chips using enzyme- or nanoparticle-catalyzed gas production as the driving force, the PT-Chip is particularly advantageous because of its robustness (i.e. strong and rapid photothermal conversion efficiency), remote on-demand controllability (i.e. precise controllability by remotely adjusting the laser parameters), stability (i.e. inertness of the photo-physical conversion efficiency to surrounding environments), and flexibility and versatility (i.e. applicability of a wide range of photothermal nanomaterials and small organic photothermal molecules). With the commercial availability of various affordable and powerful pen-style laser pointers, another key merit of the photothermal microfluidic pump is the portability by using a handheld laser pointer as the light source, compared with traditional pneumatic syringe pumps. Furthermore, unlike most traditional injector-based microfluidic pumping, no connecting accessories (e.g. injecting syringes and tubes) are required for the PT-Chips, thus optimizing the operational space and simplicity. This is the first report of microfluidic bar-chart chips using the on-chip nanomaterial-mediated photothermal effect as the microfluidic driving force, which will have great potential for various microfluidic applications, particularly for visual quantitative POCT of various biochemicals.

### 4.A.I. Materials and Methods

Materials and Instruments:

Poly(methyl methacrylate) (PMMA, 2.0 mm in thickness) sheets were purchased from McMaster-Carr (Los Angeles, Calif.). Polydimethylsiloxane (PDMS, Sylgard 184) was acquired from Dow Corning (Midland, Mich.). Graphene oxide (GO) nanosheets were the product of Graphene Laboratories (Calverton, N.Y.). Methylene blue (MB) was purchased from Sigma-Aldrich (St. Louis, Mo.). Red food dyes were obtained from Walmart (El Paso, Tex.). Prussian blue nanoparticles (PB NPs) were prepared according to the literatures. All solutions and nanomaterial suspensions were prepared with ultrapure Milli-Q water (18.2 MΩ·cm) collected from a Milli-Q system (Bedford, Mass.). Unless otherwise stated, all other chemicals were of analytical grade and used as received. UV-Vis absorption spectroscopic characterization was carried out on a SpectraMax Multi-Mode Microplate Reader (Molecular Devices, LLC, Sunnyvale, Calif.) utilizing a 96-well microplate. The temperature of the suspensions was measured by using a digital thermometer (KT-300 LCD) with a detection range of −50 to +300° C. The 808 nm diode laser (MDL-III-808) with an output power (PSU-III-LED) adjustable from 0 W to 2.5 W was obtained from Opto Engine LLC (Midvale, Utah).

### Design and Fabrication of the PMMA/PDMS Hybrid Photothermal Bar-Chart Chip

As shown in FIG. 2, three (3) chips (see Figure S1) demonstrate the versatile applications of the PT-Chips. Patterns of these chips were designed with the Adobe Illustrator CS5 software, followed by laser ablation of the PMMA/PDMS sheets by employing a laser cutter (Epilog Zing 16, Golden, Colo.). As shown in FIG. 2, the PT-Chip (Chip 1) was composed of three layers including a bottom PMMA plate (2.0 mm in thickness), a middle PDMS slice (4.0 mm in thickness), and a top PDMS sealing slice (2.0 mm in thickness). Channels with a width of 0.25 mm and inlets with a diameter of 3.0 mm were created on the bottom PMMA plate by employing the laser cutter. The fabrication parameters (e.g. speed:power intensity) for channels and inlets were 40:40% and 25:50%, respectively. An on-chip ruler for measurement of the bar-chart pumping distance was carved on the bottom PMMA plate. Inlets and outlets with a diameter of 3.0 mm were thoroughly punched in the middle PDMS slice by using a puncher (Harris, USA). Both PMMA plates and PDMS slices were thoroughly washed before the chip assembly, followed by the treatment with a plasma cleaner (PDC-32 G) for 30 seconds. To fabricate the PMMA/PDMS hybrid bar-chart chip, the bottom PMMA plate with patterns facing up was coated by the middle PDMS slice. The two layers were aligned to overlap corresponding wells. The nanomaterials (PB NPs and GO) were first suspended in aqueous solutions of the dyes (i.e. MB and RD) and then preloaded in inlets (20 μL) of the chips. The inlets were ultimately sealed with the top PDMS slice.

### Design and Fabrication of the Photothermal Microfluidic Transport Chip

The single-sample transport chip (Chip 2 of FIG. 2) was fabricated with five layers: 1) a bottom PMMA plate (2.0 mm in thickness) with superficially carved reservoirs and outlets facing up; 2) a PDMS slice (4.0 mm in thickness) as the second layer with thoroughly punched reservoirs and outlets; 3) another PMMA plate as the third layer with thoroughly carved outlets and reservoir entrances (3.0 mm in diameter), and superficially carved patterns with channels and sample wells facing up; 4) a PDMS slice (4.0 mm in thickness) with thoroughly punched sample wells and outlets as the fourth layer; 5) another PDMS slice (2.0 mm in thickness) as the top sealing layer of the sample wells. All reservoirs and outlets were in the same diameter of 6.0 mm. The diameter of all sample wells was 4.0 mm. The width of channels on the PMMA plate (i.e. the third layer) was 0.25 mm. All layers were sequentially assembled together from bottom to top by aligning corresponding wells. PB NPs suspensions (40 μL per well) were injected into reservoirs through the reservoir entrances after assembling the third layer. Target samples (20 μL), such as dyes and Au NPs, were preloaded in the sample wells after assembling the fourth layer. The multiplexed transport chip (Chip 3 of FIG. 2) was fabricated according to the same protocol as mentioned above except the major difference in one central reservoir shared with six channels in this chip. Six sample wells were connected to one central reservoir entrance.

### Off-Chip and On-Chip Investigation of Photothermal Effects of Nanomaterials

To investigate the off-chip photothermal effect of PB NPs and GO, the temperature changes of both suspensions were monitored during the laser irradiation process. Different concentrations of the nanomaterial suspensions (1.0 mL) in disposable UV cuvettes were horizontally exposed to the laser at a power density of 3.12 W/cm² for 10 min. A digital thermometer was inserted into the suspensions to monitor the temperature change. For the further quantitative off-chip photothermal investigation, PCR tubes with different concentrations of the nanomaterial suspensions (0.1 mL) were vertically irradiated by the laser at a power density of 5.26 W/cm² for 1.0 min. The temperature of the suspensions was measured by using the digital thermometer immediately after the irradiation. It should be noted that the laser irradiation intensity changed (3.12 or 5.26 W/cm²) due to different laser irradiation directions and varying surface areas in different measurement situations. To study the on-chip photothermal effect of the nanomaterials, inlets preloaded with different concentrations of the nanomaterial-dyes suspensions (20 μL per well) were individually irradiated by the laser at a power density of 2.6 W/cm² for different times. In order to record the on-chip bar-chart pumping distance, pictures of the chips were immediately taken upon the termination of the laser irradiation by using a camera (Canon EOS 600D) or a smartphone camera. The bar-chart pumping distance of the dyes was quantitatively measured by using the on-chip ruler.

### On-Chip Transport of Substances Using the Photothermal Microfluidic Pump

For on-chip (Chip 2) single-sample transport, reservoirs preloaded with different concentrations of PB NPs suspensions (40 μL per well) were individually irradiated by the laser at a power density of 2.6 W/cm² for different times. Pictures of the chips were immediately taken after the irradiation. The pumping time and pumping distance of target samples from sample wells to outlets were accurately recorded to calculate the transport rate. For the multiplexed transport chip (Chip 3), central reservoirs preloaded with PB NPs suspensions (80 μL, 0.4 mg/mL) were irradiated for 2.0 minutes at a power density of 2.6 W/cm².

4.A.ii. Results

### Off-Chip Investigation on Photothermal Effects of the Nanomaterials

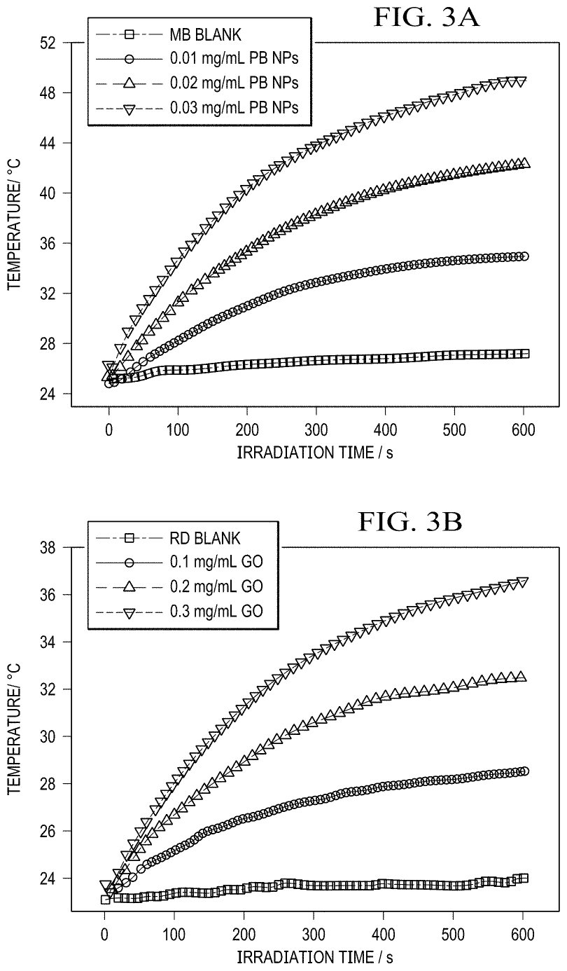

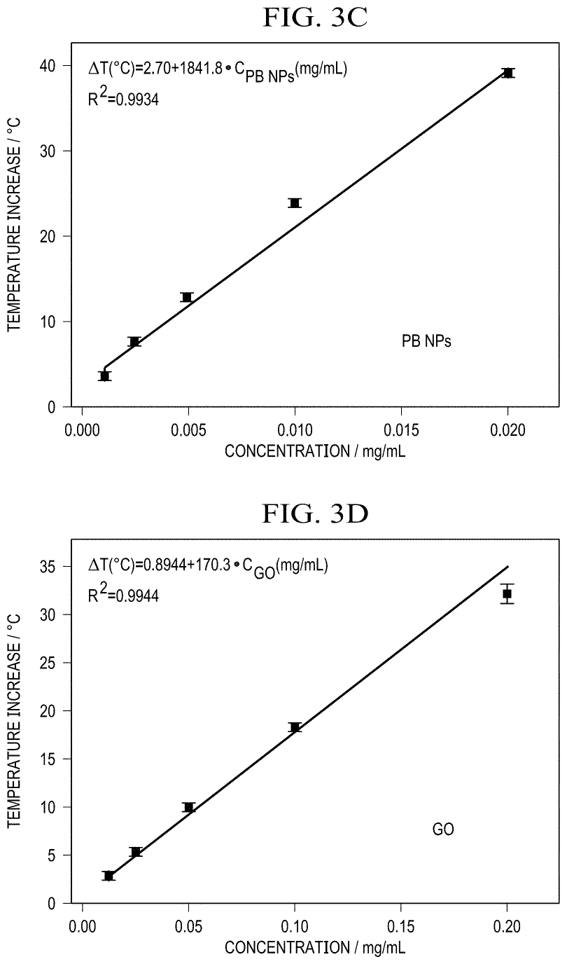

As a new generation of NIR laser-driven photothermal agent with strong photothermal conversion efficiency, PB NPs were herein selected as the model photothermal agent in contrast to a typical kind of photothermal agent, GO. In comparison with methylene blue (MB) and red food dye (RD) as the blank dye indicators, the off-chip photothermal effects of the nanomaterials in the presence of the dye indicators were investigated before the on-chip photothermal study. UV-Vis absorption spectrometry was utilized to characterize the optical absorption properties of the nanomaterials in the NIR region as shown in FIG. 3. Typically, a broad absorption band from 450 nm to 900 nm was observed in the UV-Vis absorption spectra of PB NPs with a strong absorption peak at 715 nm, which can be attributed to the charge transfer transition between Fe(II) and Fe(III) in PB NPs. Strong optical absorption of PB NPs in the NIR region after 800 nm was still observed, whereas both MB and RD showed no apparent absorption after 750 nm. As a classic kind of carbon-based photothermal agent, GO also exhibited distinct optical absorption in the NIR region but with significantly lower absorbance than PB NPs at the same concentration. The molar extinction coefficient of PB NPs at 808 nm (1.09×10⁹/M·cm) was reported to be several orders of magnitude higher than that of carbon-based nanomaterials (7.90×10⁶/M·cm).

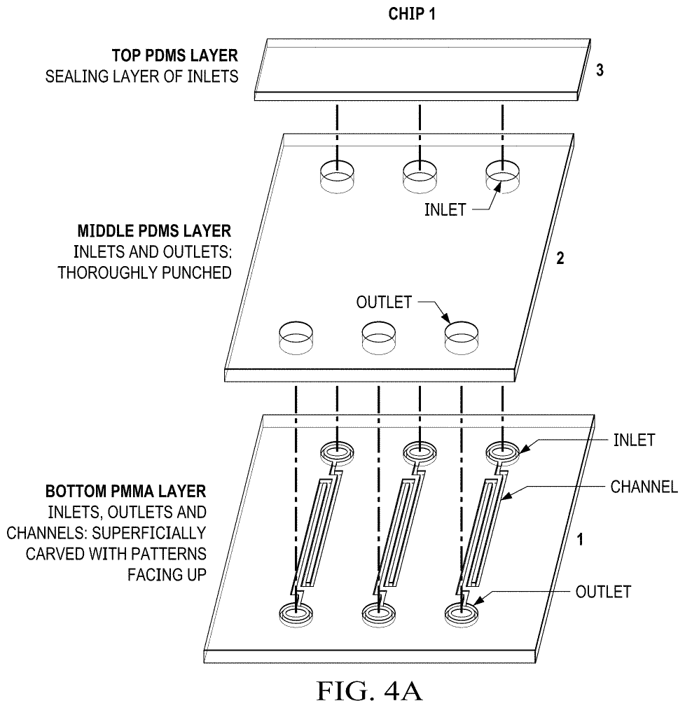

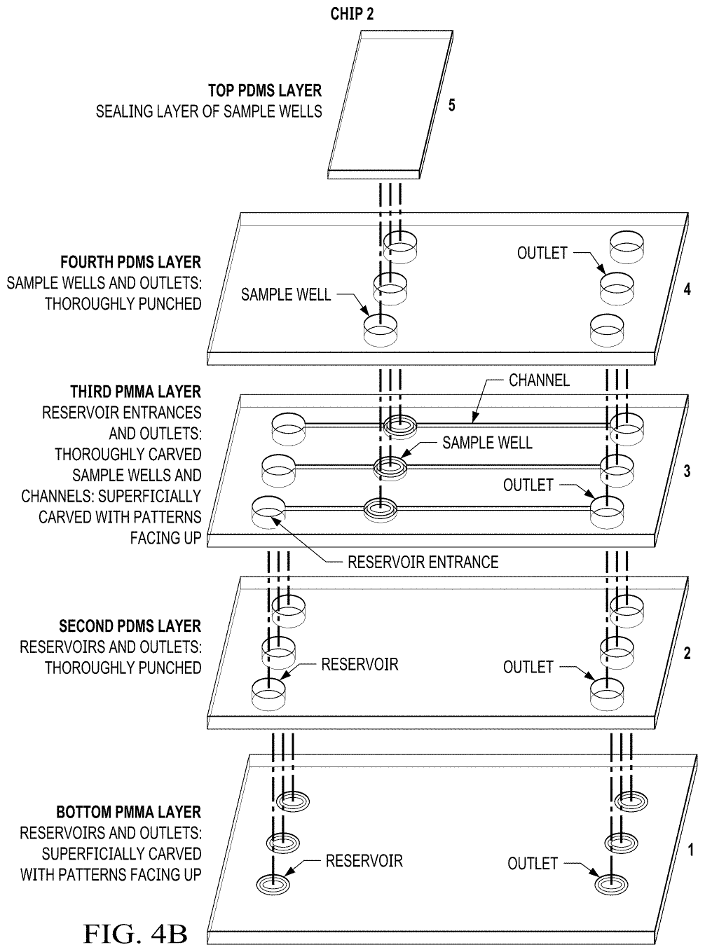

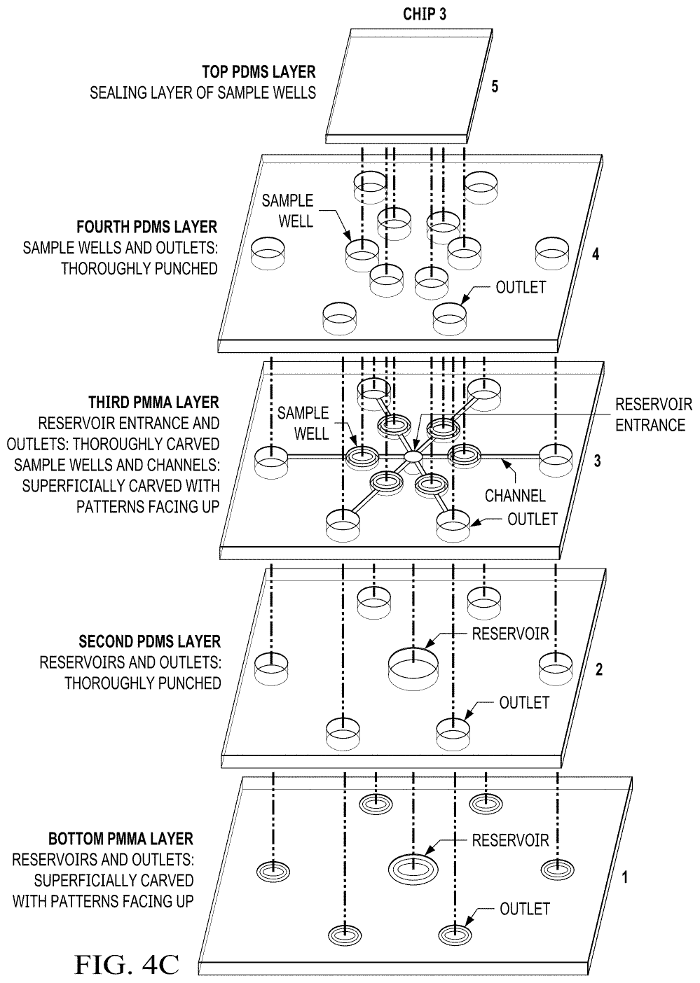

To further confirm the photothermal effect of the nanomaterials and study the effect of the dye indicators (i.e. MB and RD) on the photothermal responses, the off-chip photothermal effects of the nanomaterials in the presence of the dye indicators were investigated by employing an 808 nm diode laser as the NIR light source. FIGS. 4A-4B show the temperature changes of the nanomaterial suspensions and the blank dye solutions during the horizontal laser irradiation process of 10 min. A digital thermometer was inserted into the suspensions to record the temperature. As expected, both PB NPs and GO suspensions exhibited dramatically temperature increases in a concentration-dependent manner during the irradiation process, whereas only minor temperature increases of less than 2.0° C. were observed from the blank dye solutions. The results indicated the intrinsically poor NIR laser-driven photothermal effect of MB and RD owing to their weak optical absorption in the NIR region as demonstrated in FIG. 1. PB NPs at a low concentration of 0.01 mg/mL resulted in a drastic temperature elevation of 10.5° C. after the irradiation. Irradiation of the PB NPs suspension at a concentration of 0.03 mg/mL for only 1.0 min led to a rapid temperature increase of 5.5° C. In contrast to PB NPs, GO with a 10-fold higher concentration exhibited a much slower temperature elevation rate than PB NPs at each concentration, suggesting a stronger NIR laser-driven photothermal effect of PB NPs than GO because of the higher molar extinction coefficient of PB NPs at 808 nm.

To investigate the relationship between the photothermal effect-induced temperature elevation and the nanomaterial concentration, the nanomaterial suspensions at different concentrations were vertically exposed to the 808 nm laser for 1.0 min. The temperature of the suspensions was immediately recorded by using a digital thermometer after the irradiation. FIGS. 4C-4D show the temperature increment as a function of the nanomaterial concentration. It was found that the temperature elevation values of both PB NPs and GO suspensions were proportional to the nanomaterial concentrations in the ranges of 0.00125-0.02 mg/mL and 0.0125-0.2 mg/mL, respectively. It was worth noting that PB NPs exhibited an 8-fold higher slope than GO, further demonstrating the stronger photothermal effect of PB NPs than GO.

### On-Chip Photothermal Effects of the Nanomaterials

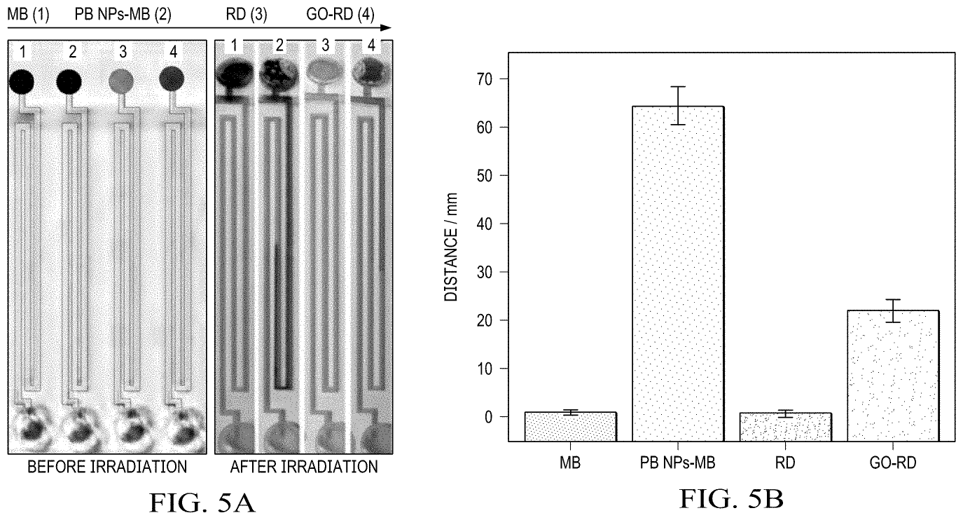

By utilizing MB and RD as the on-chip dye indicators of PB NPs and GO, respectively, the on-chip photothermal effects of the nanomaterials were investigated in a PMMA/PDMS hybrid bar-chart chip (see Chip 1 in FIG. S1). The nanomaterials were suspended in the dye solutions and then loaded in inlets of the PT-Chip, followed by sealing of the inlets with a top PDMS layer. Hence, Chip 1 preloaded with the nanomaterial-dye suspensions was assembled with only the outlets accessible to the atmosphere. To study the photothermal bar-chart microfluidic pumping performance on the PT-Chip, inlets of Chip 1 were individually exposed to the laser using blank MB and RD as the control. The bar-chart pumping distance of dye indicators was quantitatively measured by using the visual on-chip ruler. As shown in FIG. 5, both PB NPs (Channel 2) and GO (Channel 4) displayed rapid and visual on-chip bar-chart pumping of the nanomaterial-dye suspensions after the laser irradiation for 50 s (see the SI for the recorded video), whereas MB (Channel 1) and RD (Channel 3) displayed no apparent bar-chart movement due to their poor photothermal effects. GO with a 10-fold higher concentration showed a 3.0-fold shorter bar-chart movement distance than PB NPs, which can be attributed to the weaker photothermal effect of GO than PB NPs, as demonstrated in FIG. 4. In contrast, PB NPs at a low concentration of 0.1 mg/mL still resulted in a bar-chart pumping distance of 64.4 mm under the irradiation process of only 50 s, thus suggesting the robustness of the photothermal effect as the bar-chart microfluidic driving force.

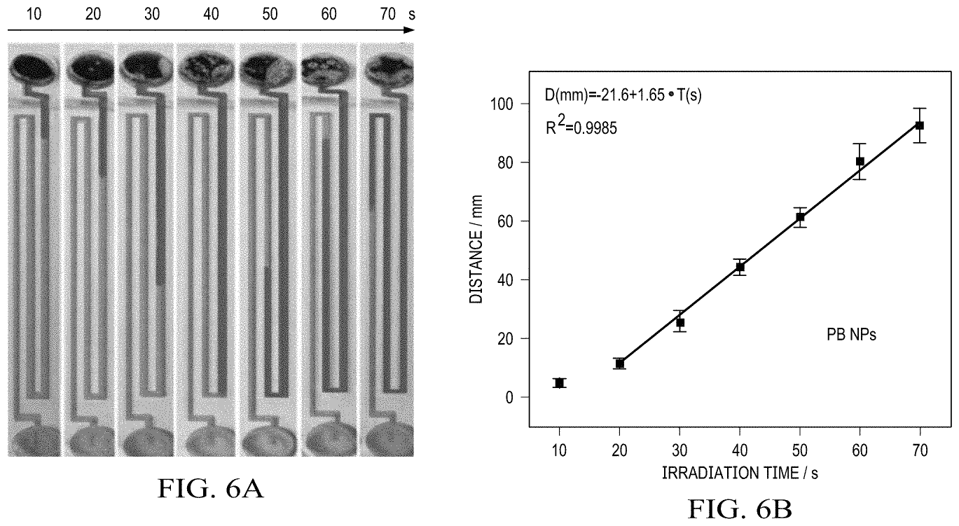

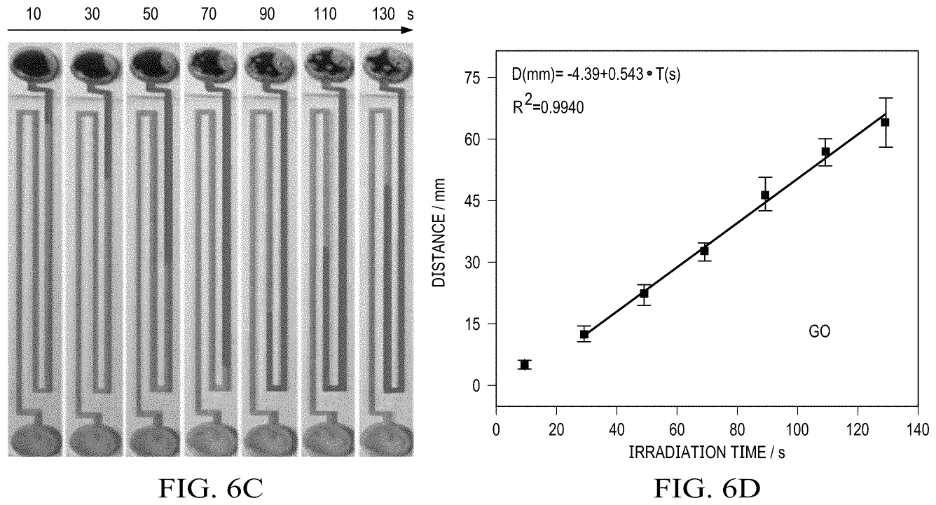

To study the influence of irradiation time on the performance of the PT-Chip, inlets of Chip 1 loaded with the same concentrations of the nanomaterial-dye suspensions were individually irradiated with the laser for different times. As shown in FIG. 6, with the increase of the irradiation time, gradually prolonged bar-chart movement distance was clearly observed in cases of both PB NPs and GO. Longer irradiation time of the nanomaterials caused the on-chip production of increasing amounts of heat, consequently leading to the progressive accumulation of increasing vapor pressure in limited volumes of the inlets. In good agreement with the result obtained from FIG. 5, PB NPs with a 10-fold lower concentration displayed a longer bar-chart pumping distance than GO at each irradiation time. Both PB NPs and GO showed a good linear relationship between the bar-chart pumping distance and the irradiation time in the ranges of 10-70 s and 10-130 s, respectively. Significantly, PB NPs exhibited a 3.0-fold higher slope than GO, implying higher bar-chart microfluidic pumping efficiency of PB NPs-mediated photothermal effect than that of GO.

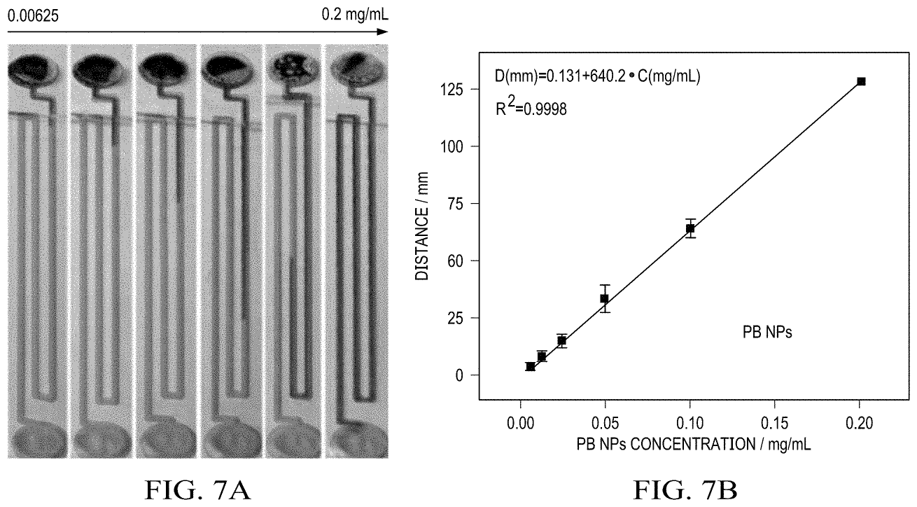

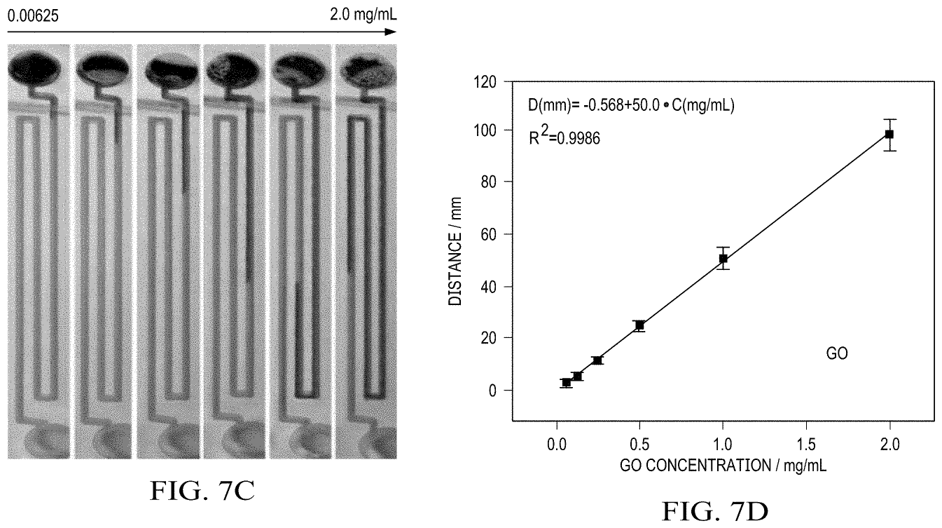

In addition, the effect of nanomaterial concentration on the performance of the PT-Chip was investigated, as shown in FIG. 7. Inlets preloaded with different concentrations of the nanomaterial-dye suspensions were individually irradiated for the same time. With the increase of the nanomaterial concentration, PB NPs displayed an increasingly prolonged bar-chart pumping distance that was obviously longer than GO even with 10-fold higher concentrations and longer irradiation times. Basically, higher concentrations of the nanomaterials accordingly led to the accumulation of higher vapor pressure in the inlets as a result of the concentration-dependent photothermal effect of the nanomaterials. The bar-chart pumping distances of both PB NPs- and GO-driven PT-Chips were proportional to the concentrations of the nanomaterials in the ranges of 0.00625-0.2 mg/mL and 0.0625-2.0 mg/mL, respectively, which laid the basis for the quantitative application of the PT-Chips. The quantitative readout results of the microfluidic pumping performance are visually displayed as on-chip ink-bar-charts without the aid of any complex and costly analytical instruments, making it particularly advantageous for POCT.

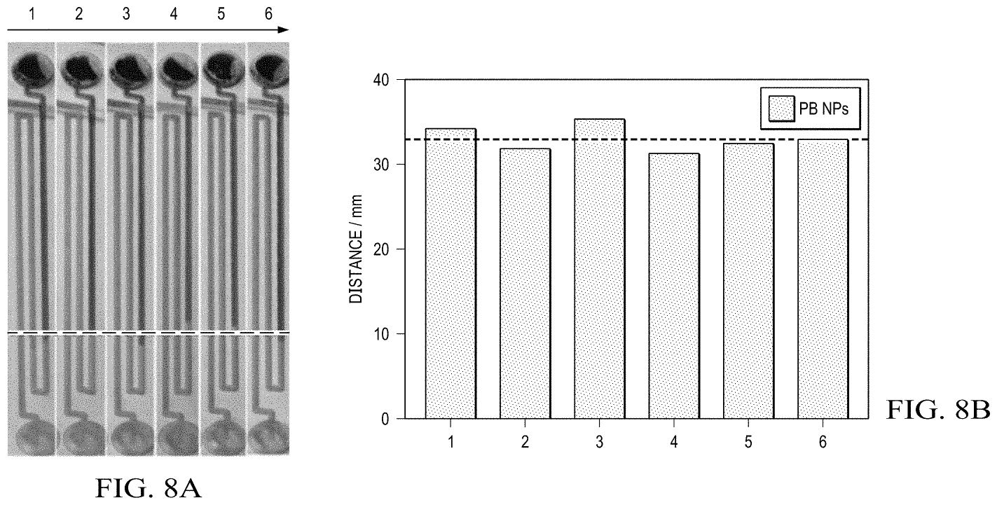

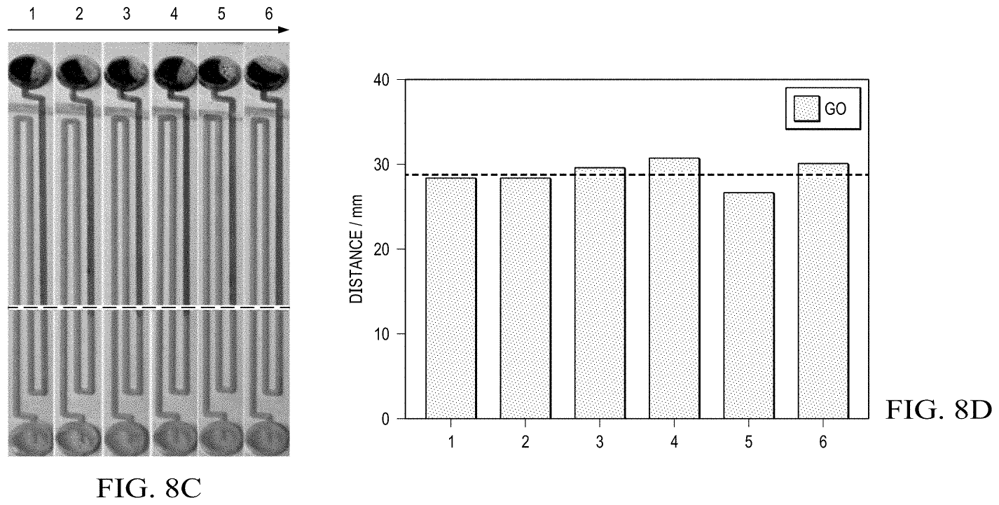

To evaluate the reproducibility of the photothermal bar-chart microfluidic pumping performance, inlets of Chip 1 preloaded with the same concentrations of the nanomaterials were individually irradiated for the same time. As shown in FIG. 8, the PT-Chips driven by both PB NPs- and GO-mediated photothermal effect displayed uniform bar-chart movement distance after the irradiation. Measurement of the bar-chart pumping distance of six parallel inlets loaded with PB NPs and GO showed low relative standard deviations (RSD) of 4.6% and 5.1%, respectively, indicating good reproducibility of the photothermal bar-chart microfluidic pumping performance on the PT-Chip.

### Multiplexed On-Chip Transport of Substances Using the Photothermal Microfluidic Pump

It has been well established that on-chip transport of fluids or substances, such as nanoparticles and chemicals, is of significant importance for various microfluidic applications. Dye transport driven by different pumping principles is the foundation of bar-chart chips. To further exemplify the application of the photothermal bar-chart microfluidic pumping system, on-chip transport of substances, such as dyes and Au NPs, using two PT-Chips (see Chip 2 and Chip 3 in FIG. 2) was conducted employing the photothermal microfluidic pumps.

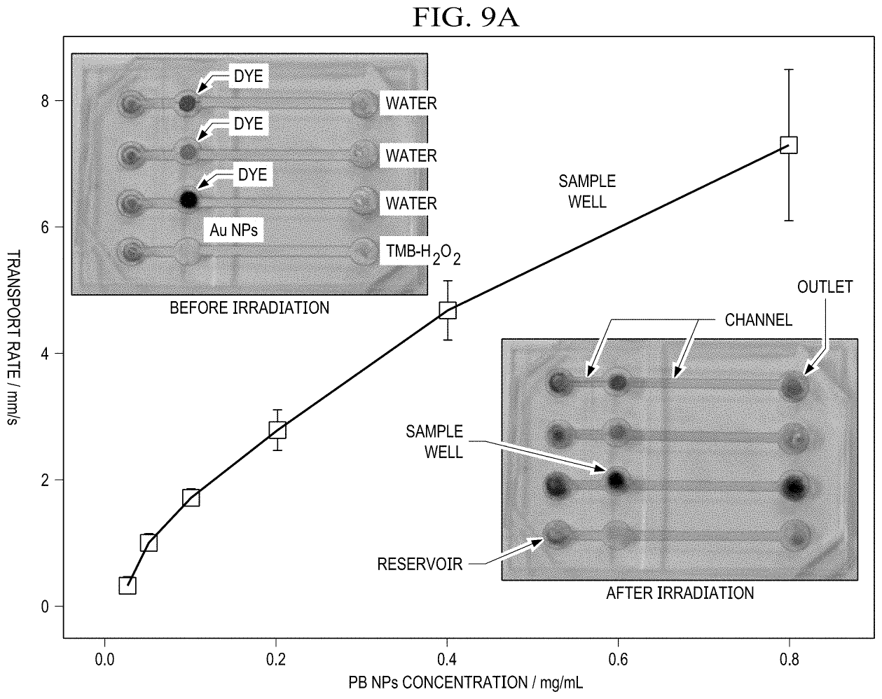

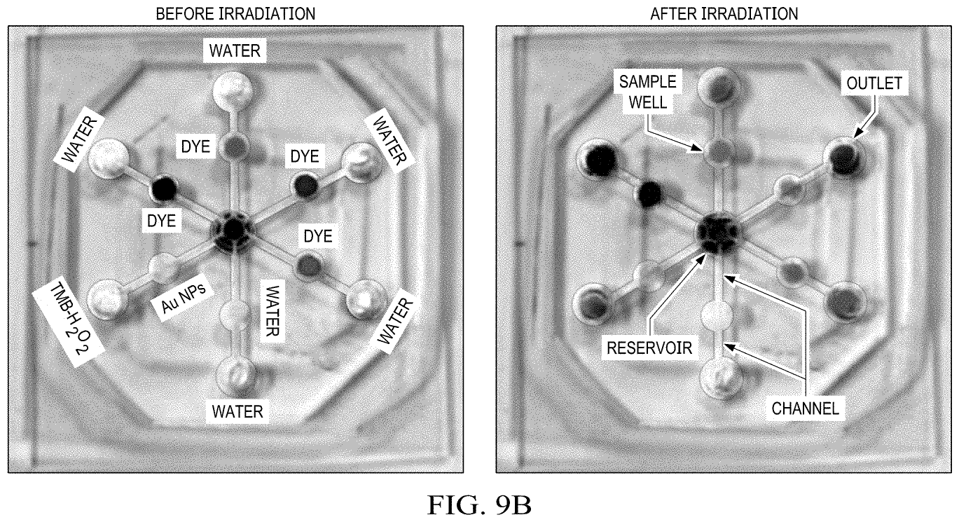

On-chip single-sample transport upon one laser irradiation process was first performed using a new microfluidic PT-Chip (Chip 2) as a proof of concept. PB NPs suspensions were preloaded in reservoirs of Chip 2 and target substances were stored in middle sample wells as shown in FIG. 9A. To assess the possibility of the PT-Chip for on-chip transport of nanoparticles, the Au NPs-catalyzed TMB-H2O2 colorimetric reaction system was integrated in the PT-Chip. Herein, Au NPs were stored in sample wells with preloaded TMB-H2O2 solutions in the outlets. Owing to the Au NPs-catalyzed TMB-H2O2 colorimetric reaction, the successful pumping of Au NPs to the outlets can result in visual color changes from colorless to blue. In order to prevent the mixing of PB NPs with target transport substances during the irradiation, PB NPs suspensions were loaded below the reservoir entrances in the third layer (i.e. the PMMA plate). Insets in FIG. 9A show the PT-Chip (Chip 2) before and after the laser irradiation of each reservoir. Upon individual irradiation of the reservoirs, dyes stored in the sample wells were pumped rapidly to the outlets through the channels, thereby displaying the corresponding color changes in the outlets. Interestingly, a clear color change from colorless to blue was also observed in the outlet loaded with TMB and H2O2, suggesting the successful pumping of Au NPs from the sample well to the outlet where the Au NPs-catalyzed TMB-H2O2 colorimetric reaction took place. A significantly high flow rate of 1.8 mm/s was achieved at a low PB NPs concentration of 0.1 mg/mL. It was found that the bar-chat flow rate was positively correlated with the PB NPs concentration in the range from 0.025 to 0.8 mg/mL. With the increase of the PB NPs concentration, increasing PB NPs-mediated photothermal effect led to the increasing vapor pressure in the reservoirs. The pressure pathway extended from the reservoir entrances to the sample wells, thus pumping target substances to move towards the outlets. These results demonstrated the successful application of the PB NPs-mediated photothermal effect as the photothermal microfluidic pump for on-chip transport of substances.

To further verify the possibility of the PT-Chip for on-chip transport of substances in a multiplexed manner, a new multiplexed PT-Chip (Chip 3) was fabricated, as shown in FIG. 9B. On this multiplexed PT-Chip, one central reservoir was connected to multiple pumping pathways and samples wells through the reservoir entrance. PB NPs suspensions (80 μL, 0.4 mg/mL) were loaded in the central reservoir, while dyes and Au NPs (20 μL per sample well) were independently stored in six sample wells. Surprisingly, upon only one irradiation process of 2.0 min to the central reservoir, the dyes and Au NPs suspensions in all sample wells were simultaneously pumped into relevant outlets with a rapid pumping rate of 60 μL/min. Hence, the PB NPs-mediated photothermal effect achieved from only one central reservoir was sufficient to pump at least six microfluidic pathways, demonstrating great potential of the PB NPs-mediated photothermal effect for on-chip sample transport in a multiplexed manner. Similarly, as discussed in the results of FIG. 5, the on-chip transport process can be immediately terminated upon the removal of the laser irradiation. The transport rate is also highly controllable by remotely adjusting the parameters of the laser. It is cumbersome to change enzyme concentrations to control the catalytic reaction rate for gas production on a highly integrated chip. Furthermore, it is worth noting that contamination and denaturation of target samples can be effectively avoided during the on-chip transport process, since target samples are well separated from the photothermal nanomaterials in the central reservoir where only vapor pressure travels outside the reservoir through the reservoir entrance. Thus, the target samples are neither contaminated nor heated during the transport process, which is of appealing advantage for on-chip multiplexed transport of biomolecules, such as protein and DNA.

### 4.B. Example 2: Photothermal and Colorimetric Immunoassay Using Transformation of Iron Oxide Nanoparticles to Prussian Blue Nanoparticles

In conclusion, a visual quantitative detection strategy for biomarkers has been developed for the first time using a novel PT-Chip. PSA as a model analyte could be detected with high specificity and sensitivity, in which the LOD of PSA spiked in human serum samples was 2.1 ng/mL. The established method could meet the cut-off requirement in the clinical diagnostics for prostate cancers. The method was further validated in real samples with satisfactory analytical recoveries in the range from 89.1% to 92.5%. Additionally, with the use of a low-cost, portable, and ready-to-use hybrid microfluidic device, the bioanalytical detection could be achieved at the point of care, eliminating the use of bulky and expensive instrumentation. The rapid and easy-to-read signals offered significant benefits, such as no need of the trained personnel. This novel introduction of nanomaterials-mediated photothermal effects into bar-chart microfluidic chips opens new opportunities towards advances in clinical bioassays, as well as for the exploration of photothermal reagents in versatile applications, such as in the diagnosis of early-stage cancers.

4.B.i. Materials and Methods

### Materials and Instruments

Chemicals and materials, including bovine serum albumin (BSA), human serum, Dulbecco's phosphate buffered saline (PBS buffer, 10 mM, pH 7.4), and prostate-specific antigen (PSA), were purchased from Sigma (St. Louis, Mo., US). Citric acid monohydrate and 1-Ethyl-3-[3-dimethylaminopropyl] carbodiimide hydrochloride (EDC.HCl) were purchased from VWR(Radnor, Pa., US). Hydrogen peroxide (H2O2, 30% w/w), N-hydroxysulfosuccinimide (Sulfo-NHS), and potassium ferrocyanide (K₄[Fe(CN)₆]) were purchased from Fisher Scientific (Hampton, N.H., US). Monoclonal mouse anti-human PSA antibody and polyclonal rabbit antihuman PSA antibody were obtained from Abcam (Cambridge, Mass., US). Fresh human whole blood was purchased from Zen-Bio (Morrisville, N.C., US) and has been tested by FDA licensed tests, showing negative results in relevant items. Iron oxide nanoparticles (Fe₃O₄ NPs with carboxylic acid groups, 30 nm in diameter) were purchased from Ocean NanoTech (San Diego, Calif., US).

Unless otherwise noted, all chemicals were used as received without further purification and Milli-Q water (18.2 MΩ·cm) was used throughout the study from a Millipore system (Bedford, Mass., US). The pH values of all buffer solutions were determined using a pH meter purchased from Fisher Scientific (Model AB15, Hampton, N.H., US).

The silicone elastomer base and the curing agent (Sylgard 184) for the fabrication of polydimethylsiloxane (PDMS) were obtained from Dow Corning (Midland, Mich., US). Poly(methyl methacrylate) (PMMA, 1.5 mm and 2.0 mm in thickness) was purchased from Mcmaster-Carr (Los Angeles, Calif., US).

### Fabrication of the PDMS/PMMA Hybrid PT-Chip

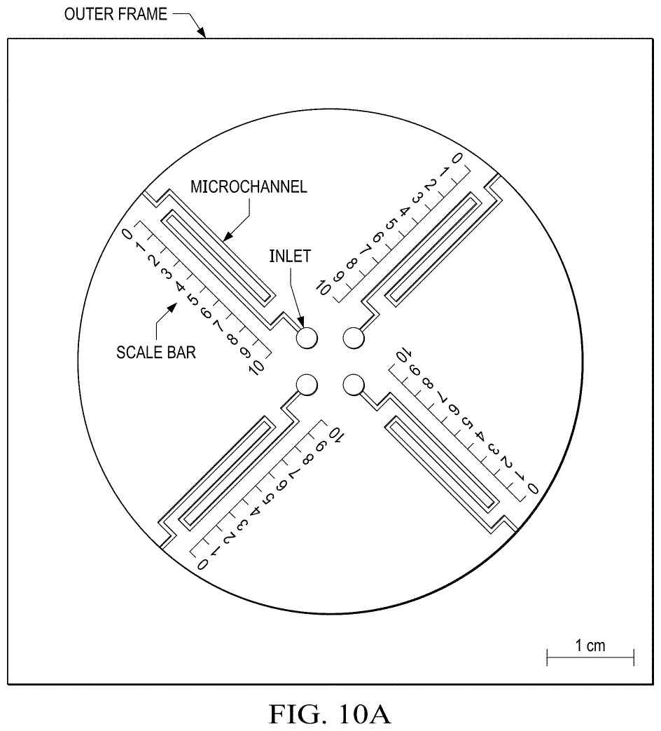

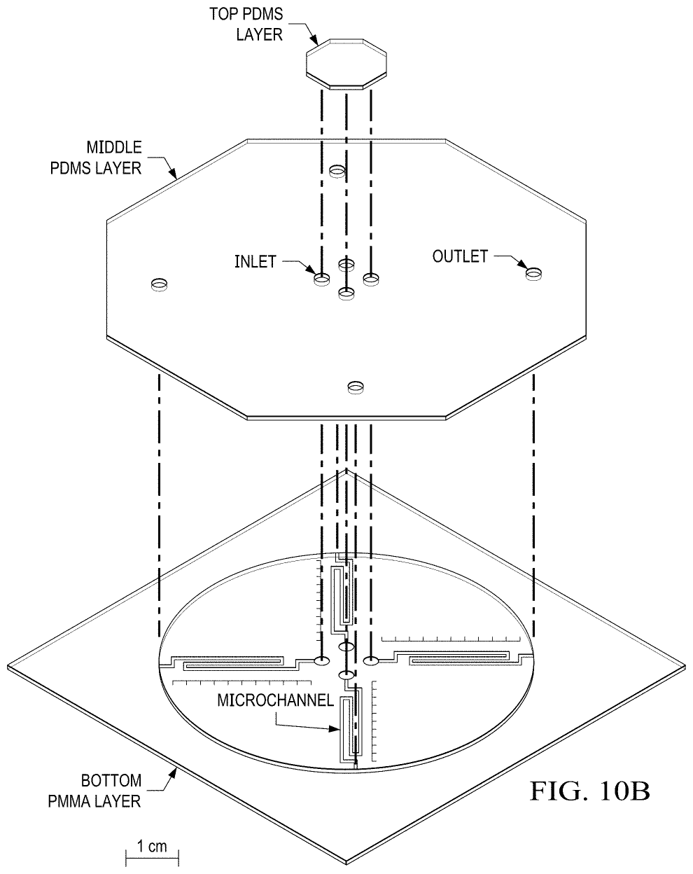

The PDMS/PMMA hybrid PT-Chip (7.5 cm×7.5 cm) consisted of three layers, including a top PDMS lid, a middle PDMS layer, and a bottom PMMA layer. All PDMS layers were fabricated according to a modified soft lithography procedure. Typically, the liquid PDMS base and the curing agent were mixed thoroughly on a mechanical stirrer (IKA RW16, VWR, Radnor, Pa., US) with a weight ratio of 8.5:1. The precursor mixture was poured into a petri dish and degassed in a vacuum desiccator for 0.5 h to remove air bubbles. The mixture was then incubated in an oven at 50° C. for 0.5 h and then peeled off from the petri dish. The obtained PDMS sheet (2.0 mm in thickness) was cut to the desired shape and punched using biopsy punches, resulting in the inlet reservoirs and the outlet reservoirs with the diameters of 0.35 cm and 0.40 cm, respectively. The patterned PMMA layer was designed with Adobe AI software as shown in FIG. 10A and fabricated using 1.5 mm thick PMMA by the laser cutter. The reservoirs were cut using the laser raster mode at the power of 50% and the speed of 25%, with a diameter of 0.35 cm and a depth of 830 μm. The microchannels were cut using the laser raster mode at the power of 40% and the speed of 30%, with the total length of 50 mm, the width of 246 μm, and the depth of 334 μm. The scale bar was customized with each unit equivalent to 2.0 mm. The frame of the PMMA layer was cut using the laser vector mode at the power of 35% and the speed of 30%.

After the fabrication of each layer, two PDMS layers and the PMMA layer were assembled reversibly based on the self-adhesion. The assembly design of the PDMS/PMMA PT-Chip was illustrated in FIG. 10B. Basically, the bottom PMMA layer was sealed by aligning with the middle PDMS layer. Then the immuno-sensing solution was introduced from the center inlets. The top PDMS layer was used to seal the inlets prior to the laser irradiation.

### Preparation of the Antibody Conjugated Fe₃O₄ NPs

The antibody-conjugated Fe₃O₄ NPs were synthesized via a carbodiimide crosslinking reaction. Particularly, Fe₃O₄ NPs with carboxylic acid groups were dispersed in water with the final concentration of 0.25 mg/mL. A 25 μL mixture containing 25.0 mg/mL of EDC.HCl and 29.8 mg/mL of Sulfo-NHS was added into the above nanoparticle dispersion and allowed to react for 40 min at room temperature under gentle shaking. Then 40 μg Polyclonal rabbit anti-human PSA antibody was then added into the nanoparticle dispersion and the pH of the mixture was adjusted to 8.0 by adding 70 μL of 50 mM NaHCO₃ aqueous solution. The obtaining solution was incubated for 2.0 h at room temperature under gentle shaking. The antibody conjugated Fe₃O₄ NPs were collected via the centrifugation of the above mixture at 11,000 rpm for 7 min. The pellet was then washed three times with PBS buffer (pH 7.4, 10 mM). Finally, the antibody conjugated Fe₃O₄ NPs were dispersed in 1.0 mL PBS buffer (pH 7.4, 10 mM, containing 0.2% BSA), and stored at 4° C. prior to use.

### Nanoparticles-Mediated Immuno-Sensing Procedures

Nanoparticles-mediated immuno-sensing was performed according to a modified procedure. Basically, 100 μL of 30 μg/mL monoclonal mouse anti-human PSA antibody was incubated overnight for 12.0 h at 4.0° C. in a PCR tube (200 μL in volume). Then 200 μL of 5% BSA blocking buffer was then added to block the unbinding sites for 0.5 h at 37° C. Different standard PSA solutions were prepared in PBS buffer containing 0.5% BSA. Solutions with different concentrations of PSA (100 μL) were incubated in the above tube for 2.0 h at 37° C., followed by thoroughly washing with PBS buffer. The polyclonal anti-human PSA antibody conjugated Fe₃O₄ NPs (100 μL) were added and allowed for further incubation for 2.0 h at 37° C. The tube carrying sandwich immuno-sensing samples was finally washed with PBS buffer thoroughly.

In the validation and specificity tests, standard PSA with different concentrations (4 ng/mL and 8 ng/mL) was spiked in both 5-fold diluted normal human serum samples and 10-fold diluted fresh human whole blood samples. Similarly, three common interfering substances, Hepatitis B surface antigen (HBsAg), carcinoembryonic antigen (CEA), and Immunoglobulin G (IgG) were chosen with 10-fold higher concentrations than PSA (up to 80 ng/mL), and used for on-chip tests, which were prepared in 5-fold diluted normal human serum samples and 10-fold diluted fresh human blood samples, respectively.

### Nanoparticles Transformation Procedures

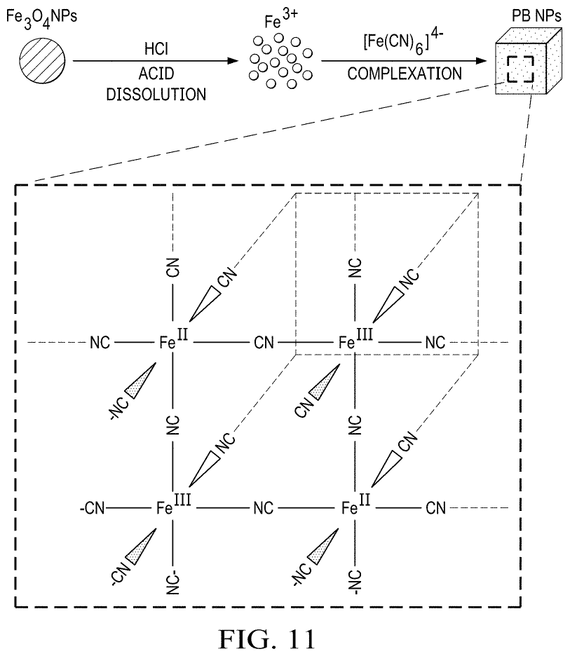

A 120 μL 0.1 M HCl solution was added to the above tube carrying sandwich immuno-sensing samples, followed by the ultrasonication for 40 min at room temperature. Then a 30 μL of 90.0 mM K₄[Fe(CN)₆] aqueous solution was used to facilitate the reaction between ferric ions and ferrocyanide ions under acidic conditions, in which Fe₃O₄ NPs captured in the sandwich immunoassay were effectively transformed to the strong photothermal reagent, PB NPs. The nanoparticles transformation reaction was depicted in FIG. 11. During the reaction, the immuno-sensing solution was mixed intensively every 10 min, and allowed to react for 1.0 h at room temperature. The procedures were characterized using UV-vis spectra prior to on-chip detection.

### Visual Quantitative Detection of PSA on a PDMS/PMMA Hybrid PT-Chip

The visual quantitative detection of PSA based on nanoparticles-mediated photothermal effect was performed under the irradiation of the 808 nm diode laser. Particularly, 4 μL of food dye solution was added into the above immuno-sensing solution for enhanced visualization of fluid flowing. Each inlet reservoir on the PT-chip was loaded with 30 μL sample solution and then sealed with the top PDMS layer. Four individual reservoirs containing sample solutions were exposed to the NIR laser at the power density of 2.21 W/cm² for 5 min. The increasing vapor pressure originated from the photothermal reagent (PB NPs), would force fluids flowing through the microchannels. As illustrated in Scheme 1, the moving distances of fluid flowing were observed and recorded directly as bar-charts on the V-chip, with the new driving force provided by nanoparticles-mediated photothermal effects.

### Working Principle of the PT-Chip

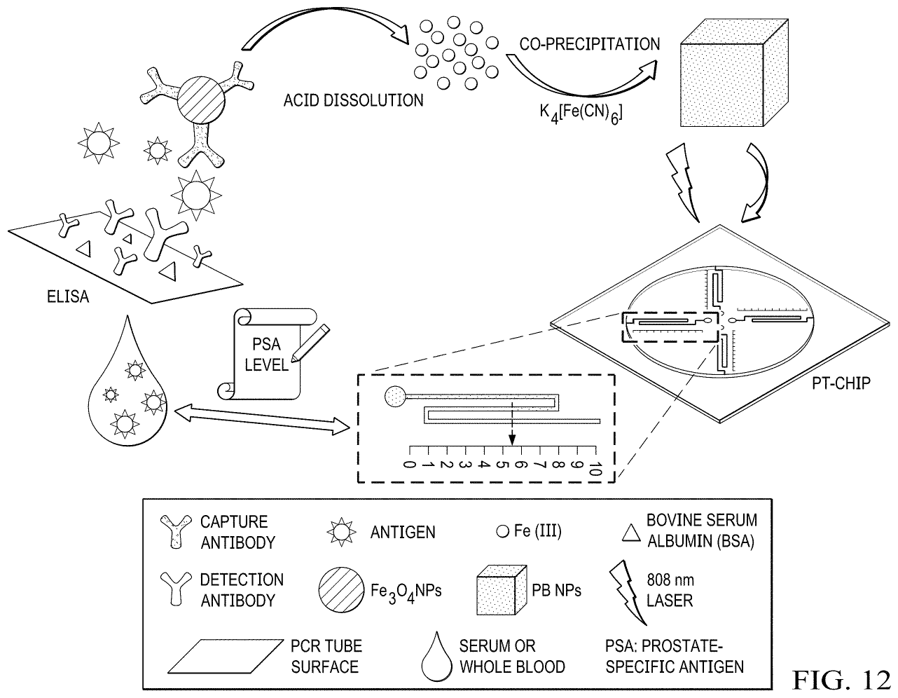

Referring now to FIG. 12, the nanomaterials-mediated photothermal effect was introduced to the visual and quantitative detection of biomarkers on the PDMS/PMMA hybrid PT-Chip. As shown, a typical sandwich-type ELISA was applied as a proof of concept, where the monoclonal antibody was immobilized on the PCR tube surface acting as the capture antibody. Prostate cancer biomarkers (PSA) were used as the target analytes, specifically binding with the capture antibody as well as the Fe₃O₄ NPs-labeled polyclonal antibody. Hence, in the presence of target, the Fe₃O₄ NPs were introduced via the sandwich structure immunoassay. The photothermal effect of Fe₃O₄ NPs has been investigated by our group and it was found that a strong NIR photothermal reagent, PB NPs could be obtained from Fe₃O₄ NPs via a simple complexation reaction due to the strong absorption in the NIR region. Basically, the captured Fe₃O₄ NPs could be dissolved in acidic solutions to release ferric ions (Fe₃+), which then reacted with the potassium ferrocyanide (K₄[Fe(CN)₆] to produce PB NPs. By using this transformation strategy of nanoparticles, a highly sensitive photothermal probe was obtained and capable to efficiently convert the immuno-sensing signals to heat under the irradiation of NIR laser. In the absence of target analytes, specific binding between PSA and capture antibodies or Fe₃O₄ NPs-labeled detection antibodies could not occur. Likewise, Fe₃O₄ NPs neither could be captured nor converted to PB NPs.

Moreover, with the assist of the ready-to-use PT-Chip, four individual experiments could be performed at the same time with only one-time laser irradiation. After sample introduction in each inlet, PB NPs with the corresponding amount obtained from the immunoassay were irradiated under the 808 nm laser. As such, the conversion from NIR light to heat was triggered via the nanomaterials-mediated photothermal effect. The heat was accumulated continuously and caused a dramatic increase in pressure on the PT-Chip, in which the pressure was transduced to drive sample solutions to move through the microchannels. Hence, the visual bar-charts movement was observed, and the quantitative detection of biomarkers could be achieved by recording the moving distance in the microchannels. It was worth noting that four scale bars (with each unit equivalent to 2.0 mm) were designed for the convenience of observation and recording of the moving distance.

4.B.ii. Results

Characterization of the Immuno-Sensing Process

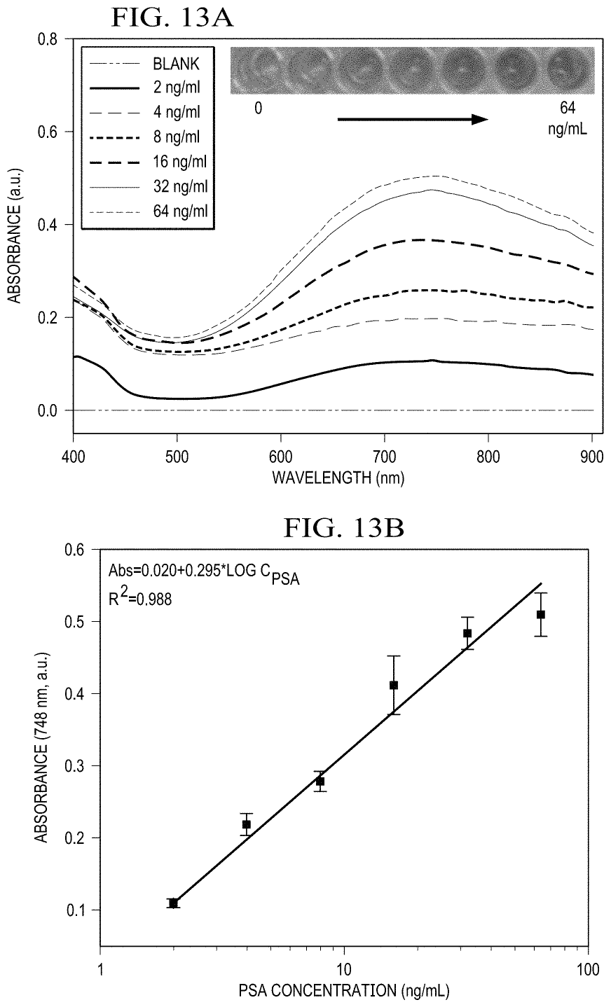

To confirm the nanoparticles-mediated immuno-sensing process, UV-Vis spectroscopic characterization was carried out on a 96-well microplate using the Microplate Reader. FIG. 13A showed the photographs and UV-vis spectra at different concentrations of standard PSA from 0 to 64.0 ng/mL in PBS buffer (pH 7.4, 10 mM, containing 0.5% BSA). After transformation of nanoparticles, it was observed clearly that the color changed from colorless or light yellow to blue while increasing the concentration of PSA in the range from 0 to 64.0 ng/mL. The maximum absorbance peaks at 748 nm proved to be the typical absorbances of PB NPs. Hence, the result verified the success of the transformation from Fe₃O₄ NPs to PB NPs, which were carried in the immuno-sensing solution.

FIG. 13B exhibited the calibration curve between absorbances and concentrations of target PSA analytes. The result turned out that the absorbances at 748 nm were proportional to the logarithmical concentrations of PSA spiking in the sandwich immunoassay. A linear relationship was established with the R² value of 0.988. The LOD in the colorimetric assay was calculated to be as low as 1.0 ng/mL based on the S/N ratio of 3, which was comparable to the commercial PSA ELISA kits (LOD: 1.0 ng/mL). The characterization of the immuno-sensing process including the transformation of nanoparticles further provided a solid foundation for the sensitive detection of biomarkers based on the photothermal effect-driven principle.

### Visual Quantitative Detection of PSA Using the PDMS/PMMA Hybrid PT-Chip

Once the immuno-sensing solution was introduced to the PDMS/PMMA hybrid PT-Chip, four individual samples were irradiated at once under the 808 nm laser. With the one-time irradiation for 5 min at the power density of 2.21 W/cm², the sample solutions started to flow through microchannels due to the photothermal effect-driven principle. The fluid flowing could be observed by naked-eyes and the yellow-colored background was used for the convenience of observation. The moving distance (ΔL) through the microchannels was recorded as the readout according to the designed scale bars.

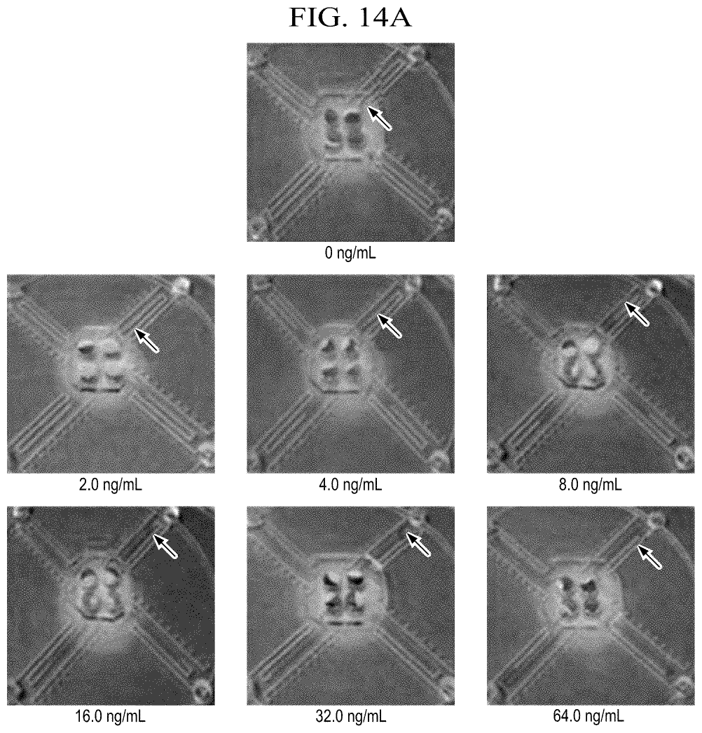

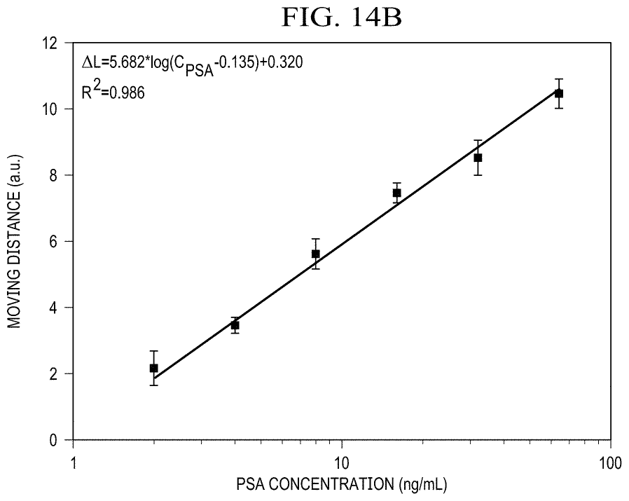

As shown in FIGS. 14A-14B, with the PSA concentrations increasing, the moving distances of sample solutions were clearly elongated under the laser irradiation. A linear relationship was obtained between the moving distances and the logarithmical concentrations of PSA in the range from 2.0 to 64.0 ng/mL, with the R² value of 0.986. The LOD was determined to be 2.0 ng/mL based on the S/N ratio of 3. The visual and quantitative detection of PSA using the PDMS/PMMA PT-Chip displayed high sensitivity and could meet the cut-off requirement of clinical diagnostics (4.0 ng/mL).

### On-Chip Visual Quantitative Detection of PSA in Serum Samples

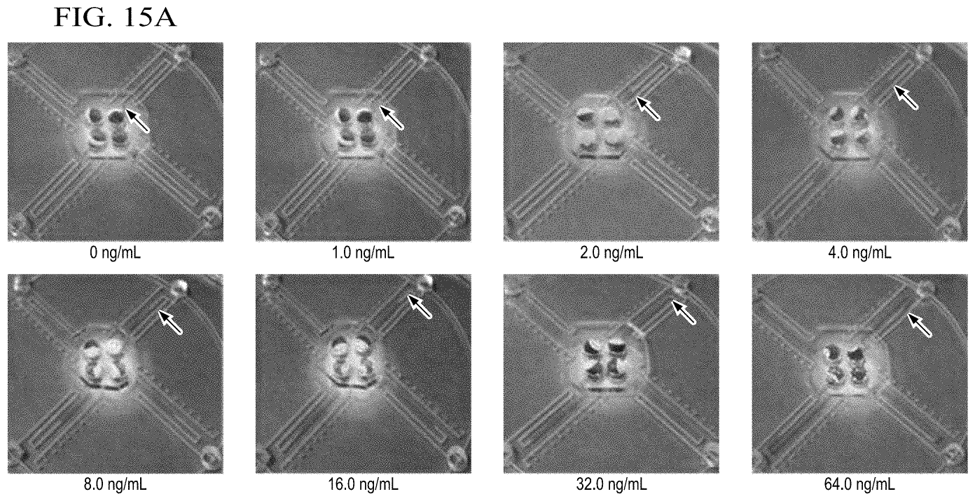

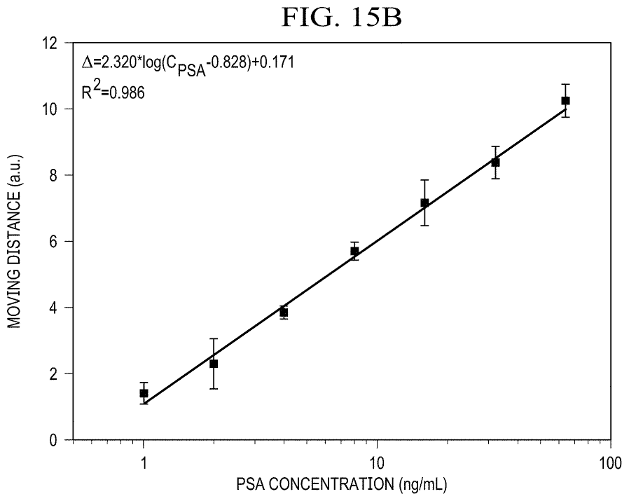

To validate this method, normal human serum samples were applied instead of PBS buffer and spiked with different concentrations of standard PSA. It is noted that 5-fold diluted normal human serum samples were applied to avoid any unspecific binding between heterophilic antibodies in real samples (e.g. serum and whole blood samples) and the capture antibodies and detection antibodies. Similarly, the typical ELISA was employed to introduce Fe₃O₄ NPs and the transformation from Fe₃O₄ NPs to PB NPs was conducted prior to on-chip detection. Under the same irradiation of a NIR laser, the moving distance (ΔL) was recorded with varying concentrations of PSA spiking in serum samples. The result in FIG. 15A showed that the moving distances of sample solutions increased as the PSA concentrations increased. The moving distance was proportional to the spiking concentration of standard PSA, with a linear relationship between ΔL and the logarithmical concentrations of PSA in the range from 1.0 to 64.0 ng/mL, with the R2 value of 0.986 in FIG. 15B. The LOD was calculated to be 2.1 ng/mL based on the S/N ratio of 3, which could meet the clinical threshold value of 4.0 ng/mL as well.

### Specificity Tests and the Evaluation of Analytical Performance

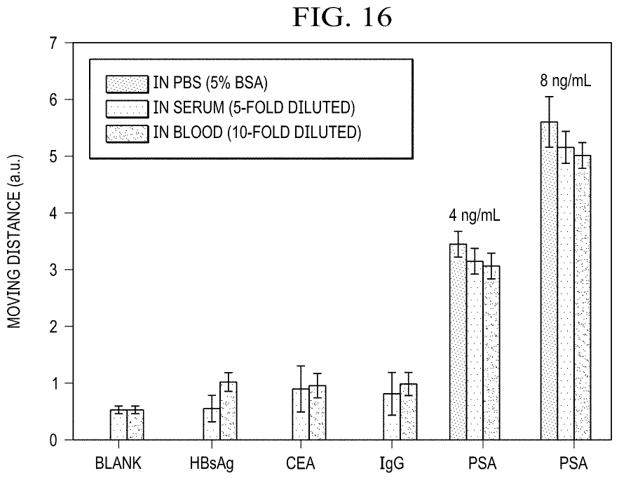

To study the specificity and further evaluate the analytical accuracy of the proposed method, 5-fold diluted normal human serum and 10-fold diluted fresh human whole blood samples were spiked with different concentrations of standard PSA. In addition, some common interfering substances, involving HBsAg, CEA, and IgG with high concentrations up to 80 ng/mL, were tested in a similar procedure. Two concentrations of PSA, 4 ng/mL and 8 ng/ml were selected according to the threshold concentration and the suspect level for clinical prostate cancer diagnostics. As shown in FIG. 16, only samples spiked with PSA had obvious moving distances, whereas no significant moving of bar-charts was observed from other interfering substances. Similar results were obtained in both serum samples and whole blood samples. The results demonstrated the high specificity and high tolerance to matrix samples of the photothermal effect-driven detection strategy using the PT-Chip.

Besides, the analytical recoveries were analyzed for the detection of spiked PSA with the cut-off level and the suspect level in both samples. By comparing the known spiking concentration in the immunoassay and the detected concentration of PSA on the PT-Chip, the analytical recovery was calculated, and the results were summarized in Table 1. It was found that the analytical recoveries with spiking concentrations of 4 and 8 ng/mL in human serum were 91.3% to 91.9%, respectively. The analytical recoveries with spiking concentrations of 4 and 8 ng/mL in fresh whole blood were 92.5% to 89.1%, respectively. Overall, all the analytical recoveries were within the acceptable criteria for bioanalytical method validation, indicating that the novel method could yield reliable results with satisfactory interpretation for the detection of PSA.

TABLE 1
Detection of PSA spiked in both human serum
samples and fresh human whole blood samples.

| | | PSA spiking | PSA detected | | |
|---|---|---|---|---|---|
| | Samp1e | concentration | concentration | Recovery | RSD |
| Matrix | No. | (ng/mL) | (ng/mL) | (%) | (%) |
| | 1 | 4.0 | 3.65 | 91.3 | 5.9 |
| | 2 | 8.0 | 7.35 | 91.9 | 5.1 |
| | 3 | 4.0 | 3.70 | 92.5 | 7.0 |
| | 4 | 8.0 | 7.13 | 89.1 | 4.5 |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of pumping fluids through microchannels of a bar-chart microfluidic chip, the method comprising:
irradiating a photothermal agent within an inlet reservoir of the bar-chart microfluidic chip with light having a wavelength that is absorbed by the photothermal agent and is converted to heat; and
responsive to an increase in vapor pressure within the inlet reservoir due to irradiating the photothermal agent, forcing fluids through the microchannels of the microfluidic chip.

2. The method of claim 1, wherein the photothermal agent is selected from the group consisting of carbon-based nanoconjugates, noble metal nanomaterials, metallic compound nanocomposites, and polymeric nanostructures.

3. The method of claim 2, wherein the photothermal agent further comprises Prussian blue nanoparticles or graphene oxide.

4. The method of claim 1, wherein irradiating the photothermal agent further comprises:
irradiating the photothermal agent with a near-infrared laser or a portable laser pointer.

5. The method of claim 1, further comprising using a photothermal pump to transport reagents in a microfluidic chip.

6. The method of claim 1, further comprising using a photothermal pump for visual quantitative detection of biochemical or disease biomarkers on a photothermal bar-chart chip.

7. The method of claim 6, wherein, a pumping distance that the fluids travel in the microchannels is proportional to an amount of photothermal agent, or a target concentration.

8. A method for quantitatively immunoassaying an analyte, the method comprising:
conjugating a sample with a photothermal agent or a photothermal precursor to form an analyte-conjugate;
loading the analyte-conjugate into an inlet reservoir of a photothermal bar-chart microfluidic chip;
irradiating the analyte-conjugate to a) increase vapor pressure within the inlet reservoir of the photothermal bar-chart microfluidic chip and b) force fluids through a plurality of microchannels; and
quantitatively determining an antibody or antigen concentration in the sample based on a moving distance that the analyte conjugate travels in the plurality of microchannels.

9. The method of claim 8, wherein the photothermal agent is selected from the group consisting of carbon-based nanoconjugates, noble metal nanomaterials, metallic compound nanocomposites, and polymeric nanostructures.

10. The method of claim 8, wherein the analyte is a protein, nucleic acid, metabolite, small molecule, fungus, virus, or bacterium.

11. The method of claim 8, further comprising:
converting the photothermal precursor of the analyte-conjugate into the photothermal agent.

12. The method of claim 8, wherein the photothermal precursor further comprises iron oxide nanoparticles and the photothermal agent further comprises Prussian blue nanoparticles.

13. The method of claim 8, wherein irradiating the analyte-conjugate further comprises:
irradiating an antibody-conjugate with a near-infrared laser or a portable laser pointer.

14. The method of claim 8, wherein the moving distance is linearly proportional to the antibody or antigen concentration in the sample.

15. The method of claim 8, wherein conjugating the sample with photothermal agent or photothermal precursor to form an analyte-conjugate further comprises:
reacting the analyte with a binding reagent, the binding reagent capable of specifically binding the analyte and forming a binding reagent/analyte complex;
contacting the binding reagent/analyte complex with a detection reagent comprising an iron oxide nanoparticle reagent that specifically binds the binding reagent/analyte complex; and
contacting the iron oxide nanoparticle reagent with a detection solution comprising a photothermal agent precursor under conditions forming a photothermal agent.

16. A photothermal bar-chart microfluidic chip comprising:
a photothermal agent contained within a reservoir of the bar-chart microfluidic chip; and
a number of micro-channels extending from the reservoir, wherein irradiating the photothermal agent with light having a wavelength that is absorbed by the photothermal agent causes an increase in vapor pressure within the reservoir and forces fluids through a plurality of microchannels, wherein a distance that the fluids travel in the microchannels is proportional to an amount of photothermal agent.

17. The photothermal bar-chart microfluidic chip of claim 16, wherein the photothermal agent is selected from the group consisting of carbon-based nanoconjugates, noble metal nanomaterials, metallic compound nanocomposites, and polymeric nanostructures.

18. The photothermal bar-chart microfluidic chip of claim 16, wherein the photothermal agent further comprises Prussian blue nanoparticles.

19. The photothermal bar-chart microfluidic chip of claim 16, wherein irradiating the photothermal agent further comprises irradiating the photothermal agent with a near-infrared laser.

20. The photothermal bar-chart microfluidic chip of claim 16, wherein a moving distance is linearly proportional to a concentration of a photothermal agent or a target.
